(19) United States
(12) Patent Application Publication
(10) Pub. No.: US 2024/0127938 A1
(43) Pub. Date: Apr. 18, 2024

(54) REFERRAL MANAGEMENT SYSTEM

(71) Applicant: Dashboard Traction, LLC, Edina, MN (US)

(72) Inventors: Brian Sammon, Edina, MN (US); Beau Jeffrey, Mendota Heights, MN (US); Aaron Rechlinger, Saint Paul, MN (US)

(73) Assignee: Dashboard Traction, LLC, Edina, MN (US)

(21) Appl. No.: 18/381,089

(22) Filed: Oct. 17, 2023

Related U.S. Application Data
(60) Provisional application No. 63/416,802, filed on Oct. 17, 2022.

(51) Int. Cl. G16H 40/20; G16H 10/60

(52) U.S. Cl. CPC G16H 40/20; G16H 10/60

(57) ABSTRACT

The present disclosure relates to a novel and advantageous referral management system, tool, and method for processing and intaking clients. More specifically, the present disclosure relates to a novel and advantageous system, tool, and method with the screening capabilities to quickly and easily process referrals or potential clients using a lockstep and reviewable process to determine which persons will become accepted clients and which will be declined according to general and organization-specific criteria.

### FIELD OF THE INVENTION

The present disclosure relates to a novel and advantageous system, tool, and method for managing referrals. More specifically, the present disclosure relates to a novel and advantageous system, tool, and method with the screening capabilities to quickly and easily process referrals or potential clients using a lockstep and reviewable process to determine which persons will become accepted clients and which will be declined according to general and organization-specific criteria.

### BACKGROUND OF THE INVENTION

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

There are variety of fields in which there is a process for intake of a person (referred to as the referral, client, or referent) to an organization. A referent is generally someone inquiring if a particular organization will be able to provide services to the referral. The referent provides a great deal of information that is used by the agency/organization to scrutinize the information to decide to provide services or not. Most every social service, human services or therapeutic service/daycare/school has a process of questions and actions that must be undertaken before the agency/organization determines if they can provide services. Current processes for such intakes are labor intensive and largely dependent on human input. The client typically calls the organization, accesses the organization online, fax, or uses a referral app. The organization asks for information from the client that may include, for example, name, insurance, what services are sought, what services may be covered, and identification of a payment source.

In most social services, healthcare services, human services, private schools, daycares, and other health or education-based organization, there is a client/patient/referent/referral intake process. Current models for this are very labor intensive and not streamlined. The amount of time large organizations waste processing clients who do not meet the organization's screening questions is huge, or even worse, the referral gets lost in a complicated paper or ad hoc process, angering the referent and the referral. The quicker an organization can process a referral to accept or decline the client, the quicker they can get more referrals and process for more of the correct fit/funding/finetuning to be able to keep the seats filled.

A complicating factor in the health care industry, for example, is that a new client does not have an established electronic medical record in which to input the requested information. The electronic medical record is not created unless and until the client is confirmed and accepted and their ability to pay is confirmed. Before the electronic medical record is created, intake personnel typically are workings with spreadsheets and paper to try to organize information about the referral. The intake personnel are trying to track a wide spectrum of information including the referral insurance, whether the organization was able to call and verify insurance, what type of program the referral is interested in, whether the referral meets the criteria for that program, whether the referral needs translators, transportation, etc., whether the referral wants (or has had) a tour, and the like.

The intake process typically is not streamlined and the intake personnel cobble together a plurality of spreadsheets, computer files, paper, notes, notebooks, or other and use ad hoc trackers. It is relatively common that some information may be missed and/or the process takes an undue length of time. The intake team may or may not have guidelines and criteria for the referrals, many times key elements are missed, and/or the process is not followed. The process may be decentralized, and the process may look different based on each intake staffs perception, by what part of the process each intake staff prefers to do, or by which parts of the process they know how to do. The documentation of each intake could be in numerous areas (paper, spreadsheets, SharePoint) and spread over a plurality (e.g., 10-15) of spreadsheets or paper documents that are very difficult to take over for processing task without extensive training and records management skills.

Key parts of the intake process that may be missed (and a client still approved) include: the referral not having verified funding, the referral not having an authorization, the referral not fitting behavioral screening criteria or other criteria for admission, the referral not being a “diagnostic fit” for the services and/or not having completed a diagnostic assessment, the referral having not had a tour of the facility, the referral not having a scheduled a start date, etc.

Current ad hoc trackers have no ability to easily compile detailed and extensive referent/referral information for marketing purposes and are virtually unable to: assess the speed and completeness of referrals, ensure that referrals do not move through the process without completing all the required process tasks, analyze the time that referrals take in each step, assess the speed and error rate of the intake staff, find bottlenecks in the intake process—all in printed form for administration to review. The last area is the ability for administration to provide intake staff accountability by requiring each step in the process to be able to track which intake staff advanced a referral to the next step in the process and which staff have not been following processes.

The amount of FTE (Full Time Equivalents) to organize and complete the intake process is daunting and requires a highly skilled and organized set of intake staff to ensure that all parts of the process are completed. Many times, the intake staff may struggle with the amount of ad hoc trackers and spreadsheets and may start taking shortcuts. The staff may get lost in the quantity of information or have a hard time moving the impromptu processes from staff to staff (different offices or location) or when intake staff turnover occurs.

The intake process, and management of referrals, is often onerous and complicated, with many people touching the referral file and little ability to systematically track where in the process a referral is, who is managing what stage of the process, or who may have made mistakes or dropped the ball during the process.

In some situations, institutions have tried to use types of Customer Relations Management (CRM) software. Especially in the medical field, these are of limited use at least because they are not designed to integrate with systems that verify insurance eligibility, and they do not address screening components tailored to behavioral health, human services, substance use disorder, and the like.

Current technology does not meet the specific needs of an intake department within a behavioral health, human services, or substance use disorder organization. There is a need in the art for a comprehensive referral management and/or client intake system and process for organizing the myriad information needed and for tracking progress through an intake process that is user friendly without extensive skill required.

### BRIEF SUMMARY OF THE INVENTION

The following presents a simplified summary of one or more embodiments of the present disclosure in order to provide a basic understanding of such embodiments. This summary is not an extensive overview of all contemplated embodiments, and is intended to neither identify key or critical elements of all embodiments, nor delineate the scope of any or all embodiments.

The present disclosure, in one or more embodiments, relates to a novel and advantageous intake system, tool, and method for processing and intaking clients. More specifically, the present disclosure relates to a novel and advantageous intake system, tool, and method with the screening capabilities to quickly and easily process potential clients using a lockstep and reviewable process to determine which persons will become accepted clients and which will be declined according to general and organization-specific criteria.

A system for referral management is provided. In some embodiments, the system includes a tool configured for one or more users to process a referral through one or more of a plurality of stages. The stages may include a new stage, a screening stage, an assessment stage, a tour stage, an authorization stage, and a queue. At the new stage, the referral is associated with a patient record. At the screening stage, the referral is asked a plurality of screening questions. At the assessment stage, a diagnostic assessment of the referral is made. At the tour stage the referral is given a tour. At the authorization stage, the referral is accepted and rejected. The referral is placed in the queue if accepted. A referral cannot progress from one stage to a next stage unless all required acts in the one stage are performed. The system further includes an active referrals graph and the active referrals graph provides a visual representation of the number of referrals in each stage.

The active referrals graph may provide a visual representation of the status of each referral in each stage. The visual representation may be via color coding. The system may further include a referrals list associated with the active referrals graph, wherein the referrals list displays detail about each referral.

Associating the referral with a patient record may comprise associating the referral with an existing client record. Asking the referral a plurality of screening questions may be done via a questionnaire and wherein the questionnaire is customizable by an organization using the system. The screening stage may further comprise screening the referral using artificial intelligence.

The user may be required to input identifying credentials when progressing a referral from one stage to a next stage. The system may track an amount of time taken for a referral to progress from one stage to a next stage.

A method for referral management is provided. In one or more embodiments, the method comprises receiving patient health information relating to a referral and inputting the patient health information into a referral management system, the referral management system having a new stage, a screening stage, and an intake stage. The method comprises associating the referral with a record during the new stage, screening the referral during the screening stage, processing the referral through the intake stage, and accepting or rejecting the referral. The referral cannot advance from one stage to a next stage until all requirements of the one stage are met. The method further comprises displaying a visual representation of the stage the referral is in. The visual representation may be a bar graph. The visual representation may be color coded.

The intake stage may comprise an assessment stage and a tour stage. Screening may be done based by asking a series of questions and wherein the questions are customizable for an organization using the method.

A user may be required to input identifying credentials when progressing a referral from one stage to a next stage. The method may further comprise tracking an amount of time taken for a referral to progress from one stage to a next stage.

While multiple embodiments are disclosed, still other embodiments of the present disclosure will become apparent to those skilled in the art from the following detailed description, which shows and describes illustrative embodiments of the invention. As will be realized, the various embodiments of the present disclosure are capable of modifications in various obvious aspects, all without departing from the spirit and scope of the present disclosure. Accordingly, the drawings and detailed description are to be regarded as illustrative in nature and not restrictive.

### BRIEF DESCRIPTION OF THE DRAWINGS

While the specification concludes with claims particularly pointing out and distinctly claiming the subject matter that is regarded as forming the various embodiments of the present disclosure, it is believed that the invention will be better understood from the following description taken in conjunction with the accompanying Figures, in which:

FIG. 1 illustrates a block diagram of an overall process of the intake system for use with a medical organization, in accordance with one embodiment.

FIG. 2 illustrates a table of stages of the intake system and process, in accordance with one embodiment.

FIG. 3a illustrates a first view of an Overview page, in accordance with one embodiment.

FIG. 3b illustrates a second view of an Overview page, in accordance with one embodiment.

FIG. 4 illustrates a Selected Referral page, in accordance with one embodiment.

FIG. 5 illustrates a Referral Details Comparison page for comparing the referral with an existing record, in accordance with one embodiment.

FIG. 6 illustrates the Overview page showing advancement of a referral from the New Stage to the Screening Stage, in accordance with one embodiment.

FIG. 7 illustrates further aspects of the Overview page, in accordance with one embodiment.

FIG. 8 illustrates a Referral Page shown after the New Stage, in accordance with one embodiment.

FIG. 9 illustrates an example Referral Details page with the Person section active, in accordance with one embodiment.

FIG. 10 illustrates an example Referral Details page with the Questionnaire section active, in accordance with one embodiment.

FIG. 11 illustrates an example Referral Details page with the Payers section active, in accordance with one embodiment.

FIG. 12 illustrates an example Referral Details page with the Contacts section active, in accordance with one embodiment.

FIG. 13 illustrates an example Referral Details page with the Contacts section active, in accordance with one embodiment.

FIG. 14 illustrates an example Referral Details page with the Logs & Notes section active, in accordance with one embodiment.

FIG. 15 illustrates an example Referral Details page with the Referral History section active, in accordance with one embodiment.

FIG. 16 illustrates an example Referral Details page with the Attachments section active, in accordance with one embodiment.

FIG. 17 illustrates a screen with Assessment Progress, in accordance with one embodiment.

FIG. 17 illustrates a Active Referrals Graph in a Referrals Overview screen, in accordance with one embodiment.

FIG. 18 illustrates a page including the Tour stage, in accordance with one embodiment.

FIG. 19 illustrates a window including a Queue Progress section and an authorization section, in accordance with one embodiment.

FIG. 20 illustrates a Rejection screen, in accordance with one embodiment.

FIG. 21 illustrates a screen including the option to withdraw a rejection, in accordance with one embodiment.

FIG. 22 illustrates an example screen for integrating a new referral, in accordance with one embodiment.

FIG. 23 illustrates general decision tree and data flow between a system for referral management such as disclosed herein and an example EHR system, in accordance with one embodiment.

### DETAILED DESCRIPTION

The present disclosure relates to a novel and advantageous data management system, tool, and method for managing referrals and/or intaking clients. More specifically, the present disclosure relates to a novel and advantageous intake system, tool, and method with the screening capabilities to quickly and easily process referrals using a lockstep and reviewable process to determine which persons will become accepted clients and which will be declined according to general and organization-specific criteria.

A comprehensive management system and process for managing, organizing, and processing the myriad information needed for assessing a referral and/or intaking a client is provided. The system and method provide a lockstep method of managing a referral for patient intake such that all required documentation is gathered and steps taken, with such documentation and steps being and tracked. The system and method are customizable for an organization's specific needs. For example, an organization can specify the required and/or the desired documentation and steps for managing a referral for patient intake. This system may be referred to herein as “Supportable” or “the Supportable referral management system.”

The system provides an end-to-end referral management process that may be used, for example, with behavioral health, human services, and substance use disorder businesses. The system and process are customizable and can be configured to fit many different organizational needs and be able to customize the process, monitor referral progress, track progress through steps in the process, and identify bottlenecks or weaknesses before they become problematic or fatal to the business. Generally, at any time during the intake process, the process can be picked up by new or other intake staff without loss in efficiency, thereby decreasing down time, decreasing loss of revenue from dropped referrals, increasing revenue from quicker processing time, relieving referent frustration, and reducing loss of referrals due to slow processing speeds or un-necessary denials. The system and process can be used to track time from an initial call to getting the client/patient/referral in the door.

The referral management system and process integrates multiple steps into a manageable and automated self-contained process. The process is quick, HIPAA compliant, and user friendly for both novice and experienced staff. An organization can customize the referral management system process to fit its specific needs. Social services, human services, or therapeutic services/daycares/schools generally have a process of questions and actions that must be undertaken before the agency/organization determines if they can provide services to a referral or client. The system may include a questionnaire for gathering required and/or desired information. The questionnaire may be customized to include organization-specific questions as well as more common general questions that are used in determining whether to provide services to a potential client.

The referral management system is a single, highly customizable system and process that eliminates the need for a high level of organization, spreadsheets, and/or ad hoc trackers. The referral management system ends the chaos, confusion, and difficulty of organizing referral/client information being received for multiple programs within an organization by email, phone calls, and/or faxes (or other). The referral management system substantially eliminates the need to use scanners, copiers, excel spreadsheets, SharePoint trackers, handwritten notes, or trackers created by each intake staff to sort and categorize information to be sorted and then to be put thru each intakes staff own process (collect required info, screen according to organization criteria, verify insurance, schedule required assessment, secure an insurance authorization, set up a tour (if needed), and then schedule a start date, or add to a wait list. Moreover, the referral management system and process tracks gathering of required information and fulfillment of required steps and identifies where such information or steps are missing or delayed.

The referral management system process may provide the ability for supervisors or administrators to monitor intake for speed and accuracy. The organization can access analytical data from the referral management system to analyze the referral management and intake process and identify possible bottlenecks in use of the process. Administrators can monitor the progress of any or all referrals as they are processed (on or off site). The self-contained referral management system also allows the intake staff to work remotely without require a VPN or elaborate security measures to work as long as they can access the high-speed internet.

The disclosed intake process consolidates previous methodology, eliminates the need to operate multiple platforms collecting required info, and provides new monitoring and optimization functionality. By facilitating customization of the system and process, intake and screening can be done according to organizational and/or service-specific criteria. In a healthcare context, the system can be used to verify insurance eligibility, schedule required assessments, secure an insurance authorization, set up a tour as needed, and scheduling a start date or adding to a waitlist (queue). In various embodiments, the referral management system and process may be used by an organization that is health related (human services, hospitals, nursing homes, home health care, mental health, substance abuse, autism, etc.), or in other industries or fields that receive complex referrals requiring processing prior to delivering a service or product.

In some embodiments, each step in the process is lockstep and may be hard wired into the system such that no steps can be avoided or sidestepped. To move a referral from one stage or step in the process to the next, an intake staff member must authorize the move to the next stage or step. Such authorization may require input of the staff member's identifying credentials, such as their name, and a time stamp to ensure that administration has visibility to the entire process to prevent intake staff from avoiding steps in the intake process. In some embodiments, progress from one stage or step to the next may be prevented, even with authorization, unless certain information is gathered or steps are taken. Progression through the process, steps taken in the process, intake contact notes, and documentation may be stored within the referral management system and monitored and analyzed for quality control. All collected information may be input into and kept by the system so there is not a need for spreadsheets, paper notes, or trackers. The system stores the information in a safe, secure, HIPAA compliant manner and can be configured to resist releases of information for medical records.

At a very high level, the referral management system, tool, and method walks a user, such as an intake personnel user, through a process wherein they input the name of a referral/referent/potential client and are prompted to do certain things in a certain order in a set, generally succinct, period of time. The user may need to input their name at various steps in the process for accountability. The system includes mechanisms for tracking, analytics, and reviewing the time it takes for a person to get from screening to assessment to verification and ultimate acceptance or rejection. The system includes graphical user interfaces displaying different information at different levels of detail during referral management for patient intake. Thee graphical user interfaces may include a referrals overview page including an active referrals graph displaying all active referrals in the system. The active referrals graph may give a visual representation of what stage or the process, and what status of each stage, each referral is in. This may be via color coding.

The process typically starts with an inquiry to an organization relating to a referral or potential client. The inquiry may be made in any suitable manner, such as with a phone call, fax, email, text, or referral form being submitted. A series of questions are asked and steps taken that assist the organization in making a decision of whether to accept or reject a potential client. These questions may include the potential client's name, age, address, health insurance provider number and group number, the desired program they would like to attend, and/or other information to qualify the person to be eligible. Some of the questions may be screening questions, the answers to which go to whether the potential client is behaviorally able to be in the level of care they are requesting. Other questions may determine if the services requested are needed or are the correct services. Yet other questions may verify health insurance coverage and insurance authorization needs. As information is input into the system, it can be verified—the system can explore backgrounds, demographic information, payment sources, etc.

As the intake staff proceeds through the process, the results of each stage of the process may be displayed or otherwise accessed so an administrative user can monitor speed of the process. A penultimate stage of the process may be a diagnostic stage that requires a diagnostic tool (diagnostic assessment, test, interview, other determination tool, etc.), after which the ultimate decision stage wherein the decision to accept or reject the referral may be made. If the referral is accepted, the intake staff places the referral with an open staff or into a waiting queue to be placed later. Upon acceptance, the client's information (now residing in the referral management system) can be added to an electronic health record (EHR) or official file. If the referral is declined, the referral information is not be added to the HER, the time adding to the EHR is not wasted, and the record of the denied clients will not take up valuable space in the EHR.

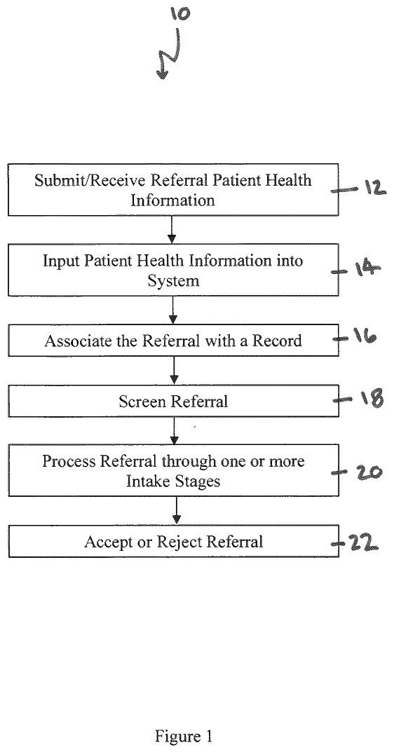

FIG. 1 illustrates a block diagram of an overall process 10 of the referral management system in accordance with an embodiment for use with a medical organization. It is to be appreciated that during the process, the referral management system collects data that can be used for monitoring or reporting purposes. Patient health information (PHI) is submitted regarding a referral or potential client (“Referral Patient Health Information”) 12, for example using a referral form. This is received by the organization. The PHI is entered into the system and is guarded in a HIPAA compliant manner 14. The referral is associated with a record 16. This may comprise matching the referral with existing people in a database for building client history, matching the referral against existing clients to ensure that existing clients are not duplicated, and/or creating a new record for the referral. The referral is screened through dynamic data collection and system behavior 18. The referral proceeds through stages of the system by meeting specific criteria 20. Insurance is verified, typically within a matter of seconds instead of hours. The system accepts or rejects the referral 22. If accepted, the referral is placed in queue for when ready to start services. In some embodiments, the system may have an add-on that connects into a MHR/EHR/EMR to pull information from the referral management system and directly add it to the MHR/EHR/EMR. Reference is made herein to the process of onboarding. Onboarding is when an organization customizes the system to their particular specifications for their particular use. While the majority of customization may be done during onboarding, it is to be appreciated that many aspects of customization may be updated during use.

In some embodiments, artificial intelligence (AI) may be used with the system and method for referral management. The AI may be used to further automate the intake process. More specifically, the system and method may include a machine learning algorithm to identify referrals or subsets of referrals who may be more likely to be accepted or rejected as clients. Training sets may be provided and developed for growing the capabilities of the machine learning algorithm. Alternatively, or additionally, the training sets may be developed organically from use of the system. AI may be used to focus on, or prioritize screening in, clients with whom an organization is most successful in treating and/or specializes in working with. Machine learning may be used to train the AI over a plurality of records. The referral management system thus can increase success rates by helping to focus intakes on organizational specialization. The referral management system further can be used to increase retention of client by screening out problematic behavior. In some cases, such screening may be done automatically and early in the process before human time is spent on the referral.

In some embodiments, the system may include an ability to track speed, accuracy, and error rate of multiple intake staff and the ability to print out or analyze results for reviews, improvement projects, or employment records. The system and its data may be leveraged to provide detailed marketing data of referent sources. In some embodiments, the system may provide detailed analysis of rejected clients to provide insight into possible growth opportunities for the organization.

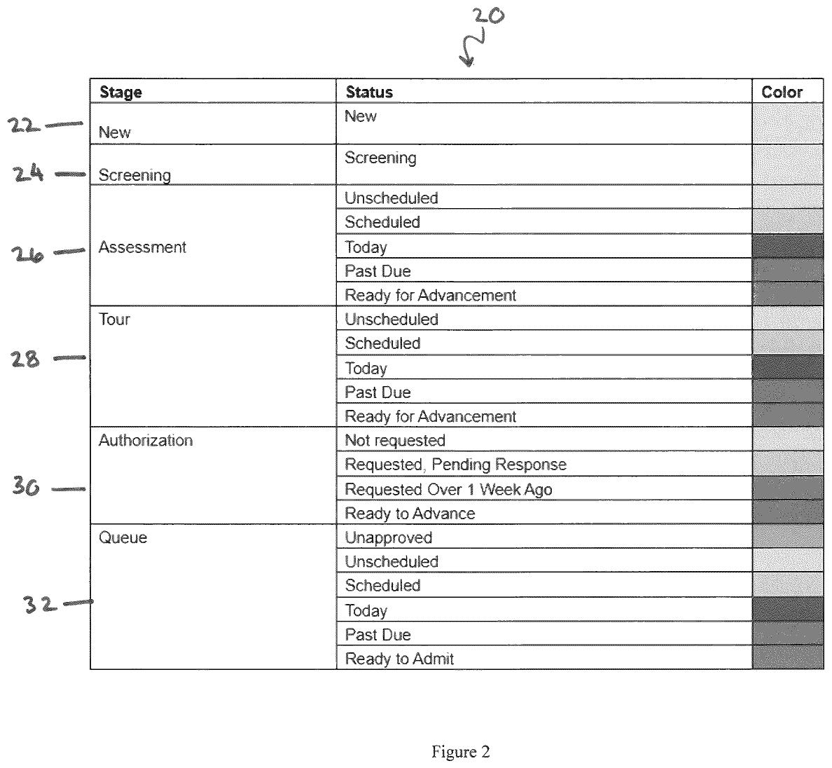

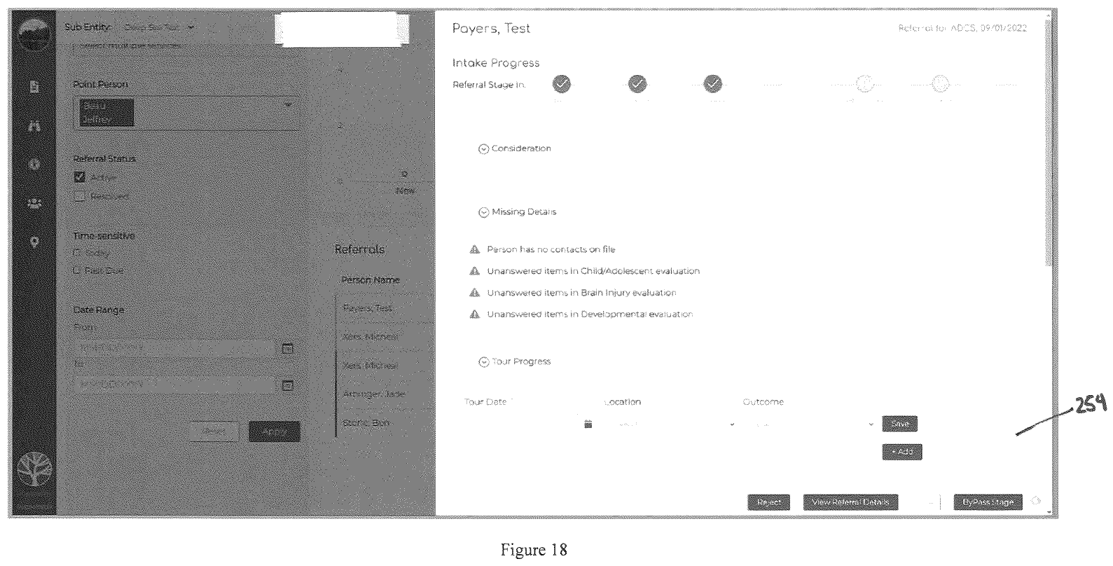

Detail will now be given of various stages or the referral management system and process. FIG. 2 is block diagram 20 illustrating possible stages that may be incorporated into the referral management system and process. These may include a New Stage 22, a Screening Stage 24, an Assessment Stage 26, a Tour Stage 28, an Authorization Stage 30, and a Queue 32. The diagram includes color coding of each status of each stage. This color coding may be used with an active referrals graph such as described with respect to FIG. 18. It is to be appreciated that not all of these stages may be used and/or additional stages may be used.

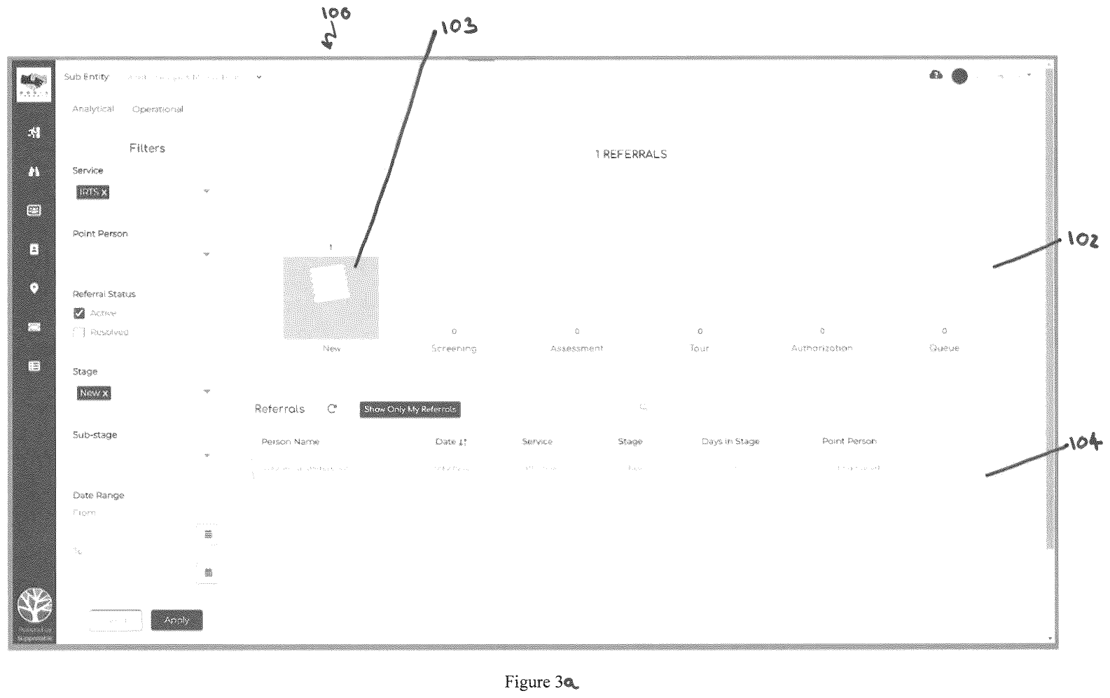

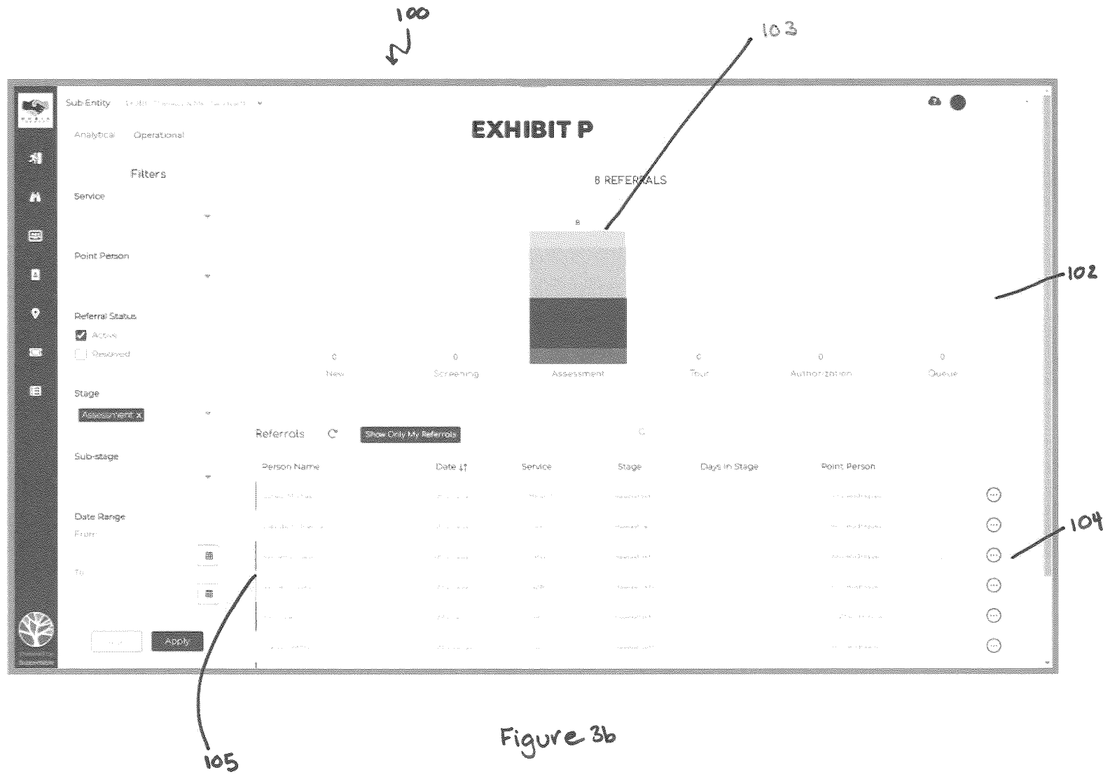

FIGS. 3a and 3b illustrates example instances of a Referrals Overview page 100. The Referrals Overview page 100 includes an Active Referrals Graph 102 and a table of referrals or referrals list 104. The Overview page 100 gives a snapshot view of all of the referrals presently in the system. The Active Referrals Graph 102 includes an indicator at each stage in which there are active referrals. The indicator maybe, for example, a bar 103. The Active Referrals Graph 102 thus shows how many referrals are at each stage of an intake process—including, for example, the New Stage, the Screening Stage, the Assessment Stage, the Tour Stage, the Authorization Stage, and the Queue.

The Active Referrals Graph may be configured to update in real time to display referrals in their various states. Such display may comprise a plurality of colors with each color representing a stage-specific state. This provides a comprehensive and clear overview of all current referrals. In the New Stage, status is new. In the Screening stage, status is screening. In the Assessment stage, status may be unscheduled, scheduled, today, past due, or ready for advancement. In the Tour stage, status may be unscheduled, scheduled, today, past due, or ready for advancement. In the Authorization stage, status may be not requested, requested/pending response, requested over 1 week ago, or ready to advance. In the Queue stage, status maybe unapproved, unscheduled, scheduled, today, past due, or ready to admit. Each stage may have an associated checklist for guiding progress through the stage and to the next stage. Each checklist can be unique to a service and/or a stage and may be configured during the onboarding process. Checklists relate to information at each stage. The completion of a checklist for a respective stage can be configured as a requirement for advancing a referral to the next stage.

The Active Referrals Graph 102 includes an indicator 103 at each stage in which referrals are currently being processed. In the example shown in FIG. 3a, one referral is shown in the New Stage. In the example shown in FIG. 3b, eight referrals are in the Assessment Stage. The indicator may further be color coded to indicate at what status of each stage each referral is at. The colors may correspond with the colors identifying statuses of each stage in the block diagram 20 of FIG. 2. Referring again to the example of FIG. 3b, referrals are shown in 4 different status of the Assessment Stage.

The referral list 104 lists each of the referrals currently in the system and can provide a table view of certain information associated with each referral. As shown, each referral may comprise one or more separate lines on the list. Information provided for each referral in the list may include, for example, a date of referral, services being sought, process stage, days in the process stage, and point person. The list may further include a color 105 associated with each referral, the color indicating the status and stage of the referral. The information on the referral page may vary depending on the stage the referral is in. If a plurality of services are sought, these may be listed separately or grouped together. In the embodiment shown in FIG. 3a, the services are grouped together and a single line for the referral is shown at the New Stage.

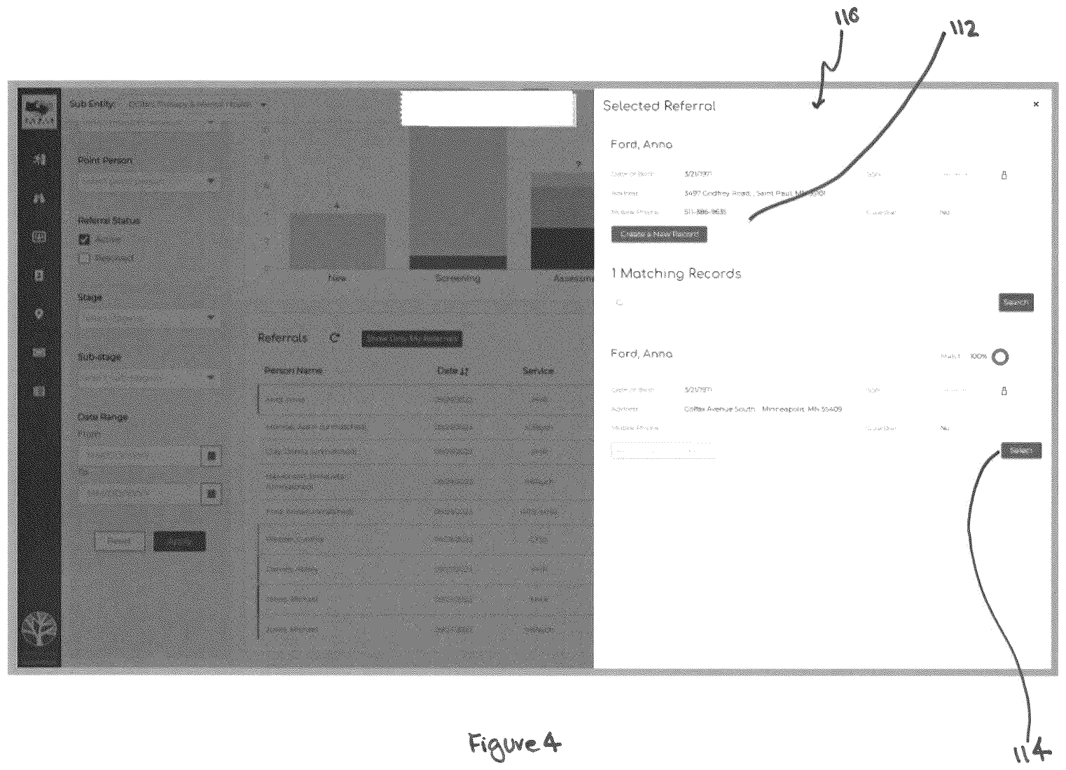

A user can click anywhere on a referral line to start processing the referral. FIG. 4 illustrates a Selected Referral page or window 110 that is made active upon selection of a referral, for example from the referral list 104 of the Overview page 100. As shown, upon selection of a referral, a user can either create a new record 112 or match the referral with an existing record 114. The system uses identifying information about the referral to search for similar records. Such identifying information may include date of birth, address, mobile phone number, social security number, or guardian. Such searching may be done automatically. Alternatively, or additionally, a user may run a further search. This may be done, for example, if the user wants to search a nickname or the like.

The user then can either create a new record for the referral and associate the referral to the newly created record or can match the referral to an existing record for a person in the database. In general, a new record is created if the referral has never been referred to the organizational entity previously. If the referral has a record with the organization, the referral can be matched to that record based on some combination of name, date of birth (DOB), social security number, and address. The system can offer suggested matching records and/or the user can search records for possible matches.

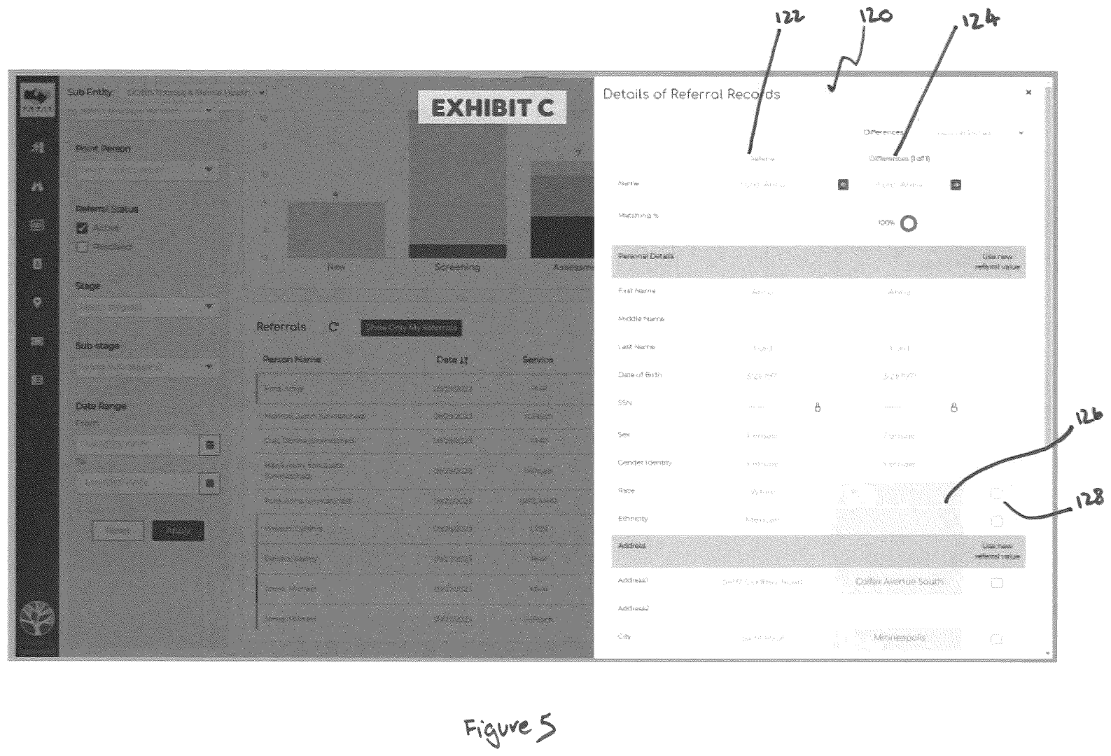

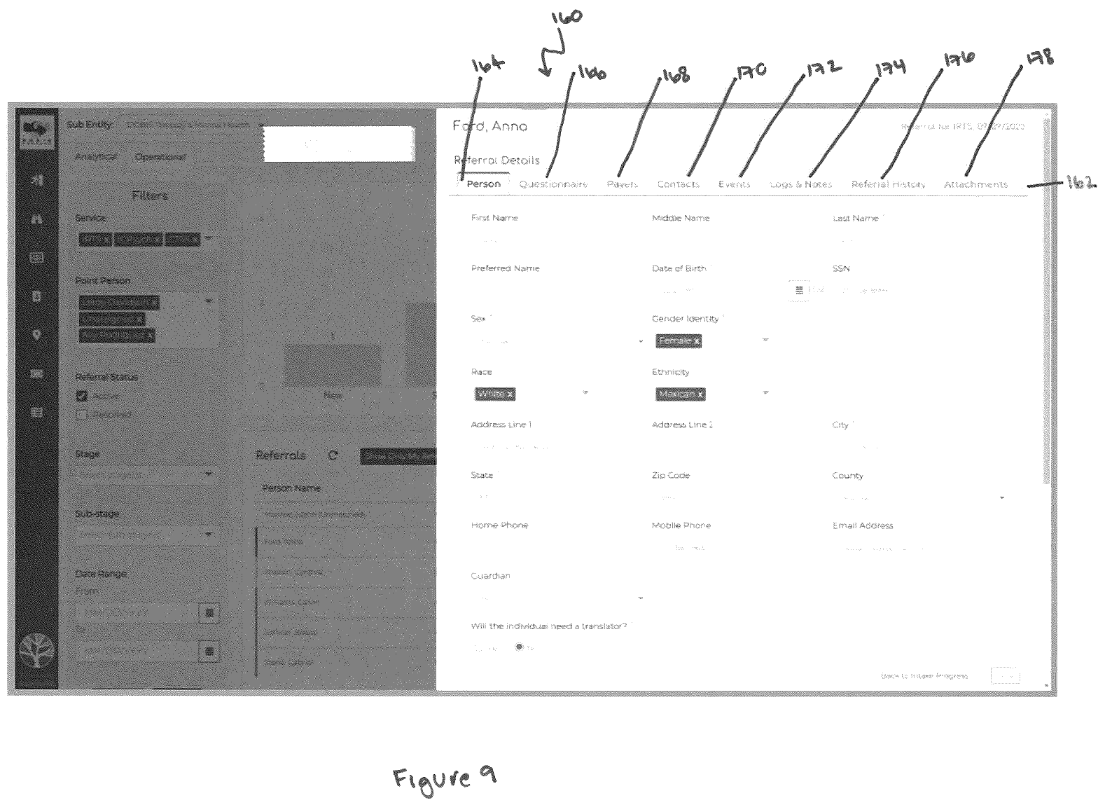

FIG. 5 illustrates a Referral Detail Comparison page 120 for comparing the referral with an existing record, in accordance with one embodiment. This page may be launched if a user chooses to match the referral to an existing record. As shown, the screen 120 displays details of the referral 122 and details of an existing record 124 record side-by-side. This allows the intake personnel user to compare values. The user can choose to show all values, only similarities, or only differences. In some embodiments, where there are differences between values in the referral and values in the existing record, these differences may be flagged, for example by accentuating or highlighting the differences 126. If the user chooses to match the referral to an existing record, the user may select values from the referral to use to update the record 128. The user may choose to update all values wherein there are differences or may choose to update only select values. FIG. 9, discussed below, illustrates referral details that may be updated.

After the referral is associated with a new record or with an existing record, the referral passes from the New Stage to the Screening Stage.

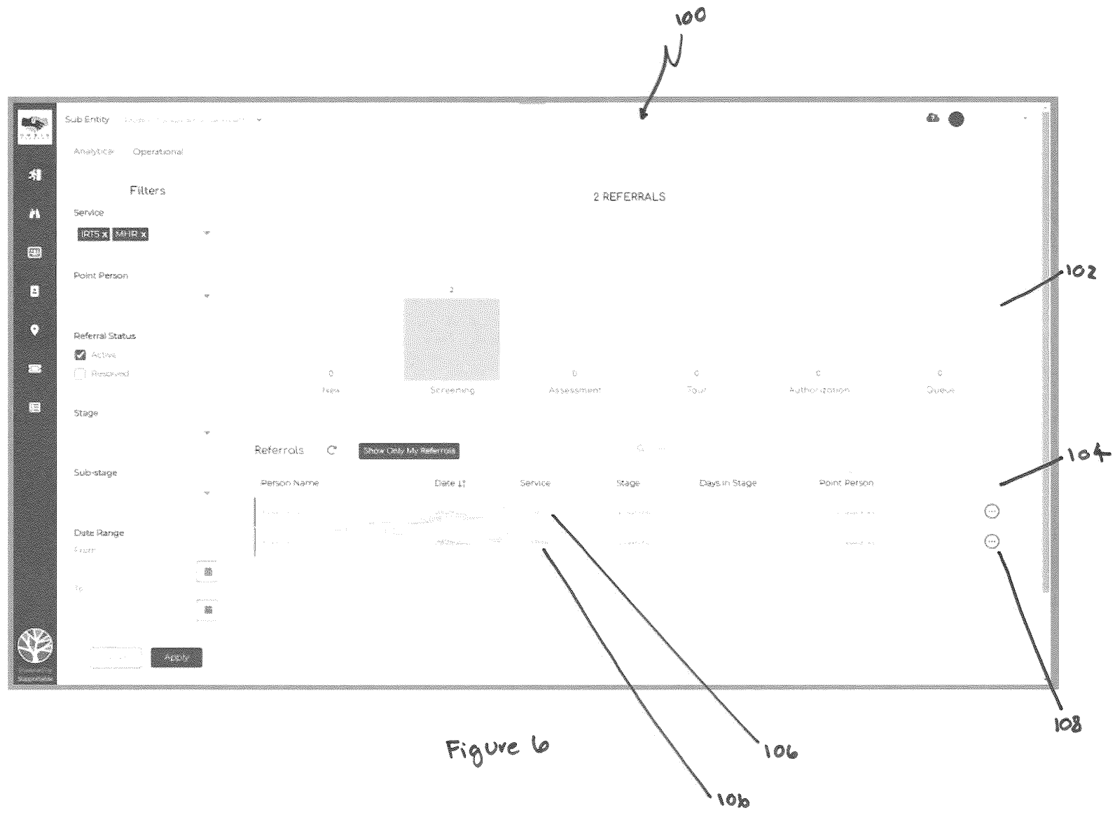

FIG. 6 illustrates the Overview page 100 showing advancement of a referral from the New Stage to the Screening Stage on the Active Referrals graph 102, in accordance with one embodiment. As shown, at this stage, each service 106 may be split into a different line for the referral within the list 104. This permits a user to process the referral differently for each service such that each instance of the referral can take a different path through the intake process. A user may take the referral through different paths through the intake process defined by a specific service when configured in an admin portal. In alternative embodiments, the plurality of services for the referral may be grouped into a single line on the list 104.

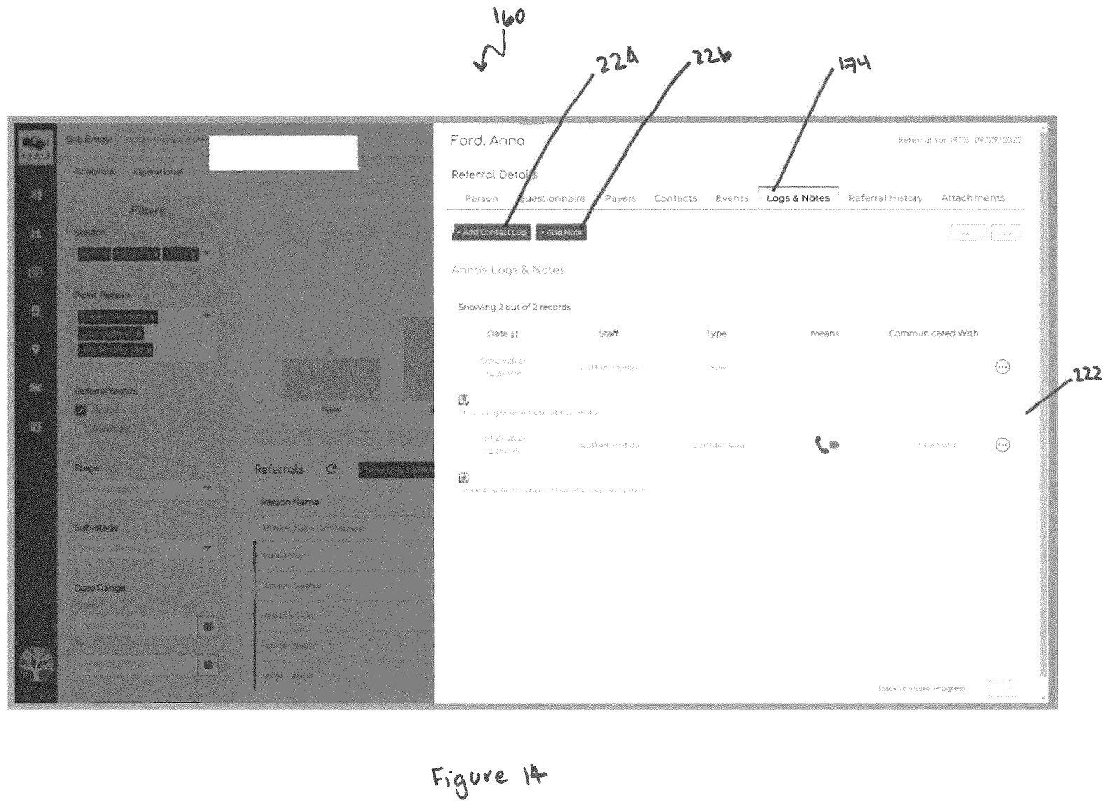

A quick-action selection may be provided to the user. In the embodiment shown in FIG. 6, the user may click on the ellipsis 108 to select quick-action. The quick action may include “Add Note,” shown in FIG. 14, “Add Contact Log,” shown in FIG. 14, Assign to Point Person (choose from a list of users with applicable permissions), or Reject, for example.

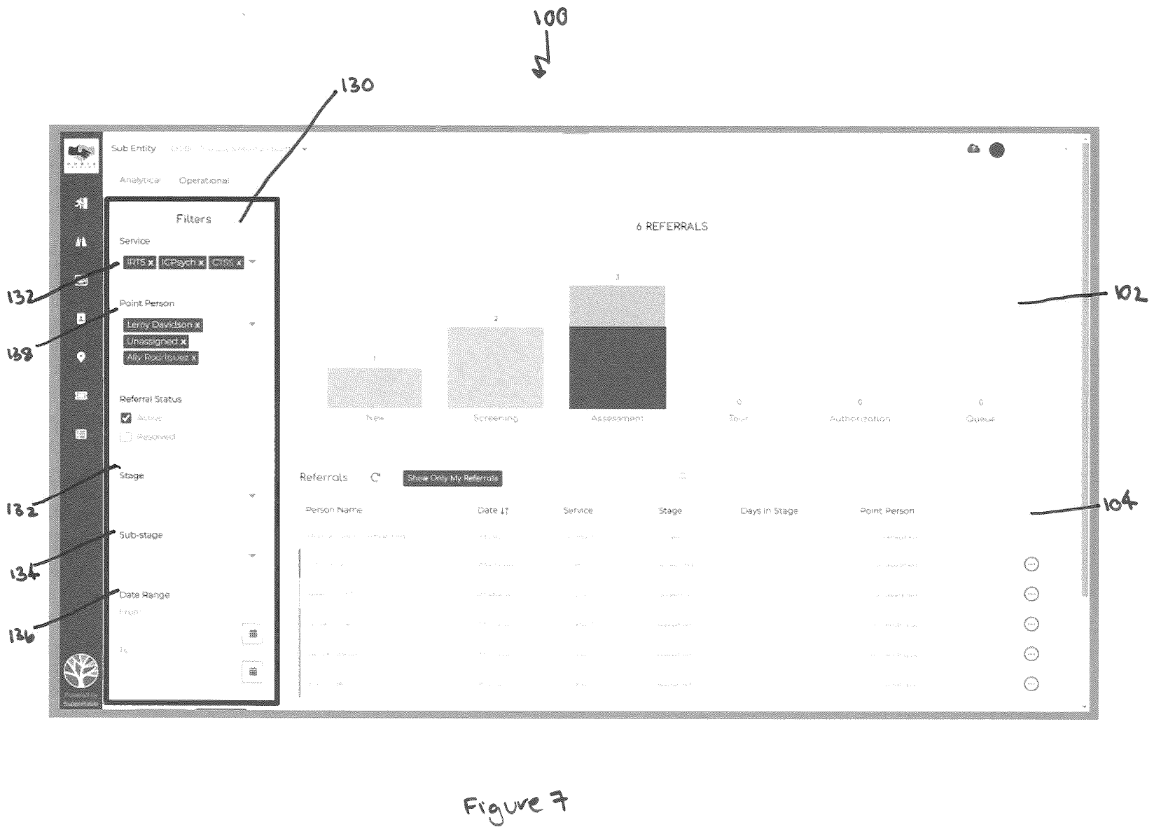

FIG. 7 illustrates further aspects of the Overview screen 100, in accordance with one embodiment. More specifically, FIG. 7 illustrates filter options 130 for filtering the referrals shown in the Overview screen 100. As shown, a user may be given an option to filter referrals by various parameters such as service, stage, sub-stage, date range, point-person, etc. The values for the filters may be manually input or selected from a drop-down The a Active Referrals graph 102 and a referral list 104 can actively update to reflect only referrals matching the selected filter(s).

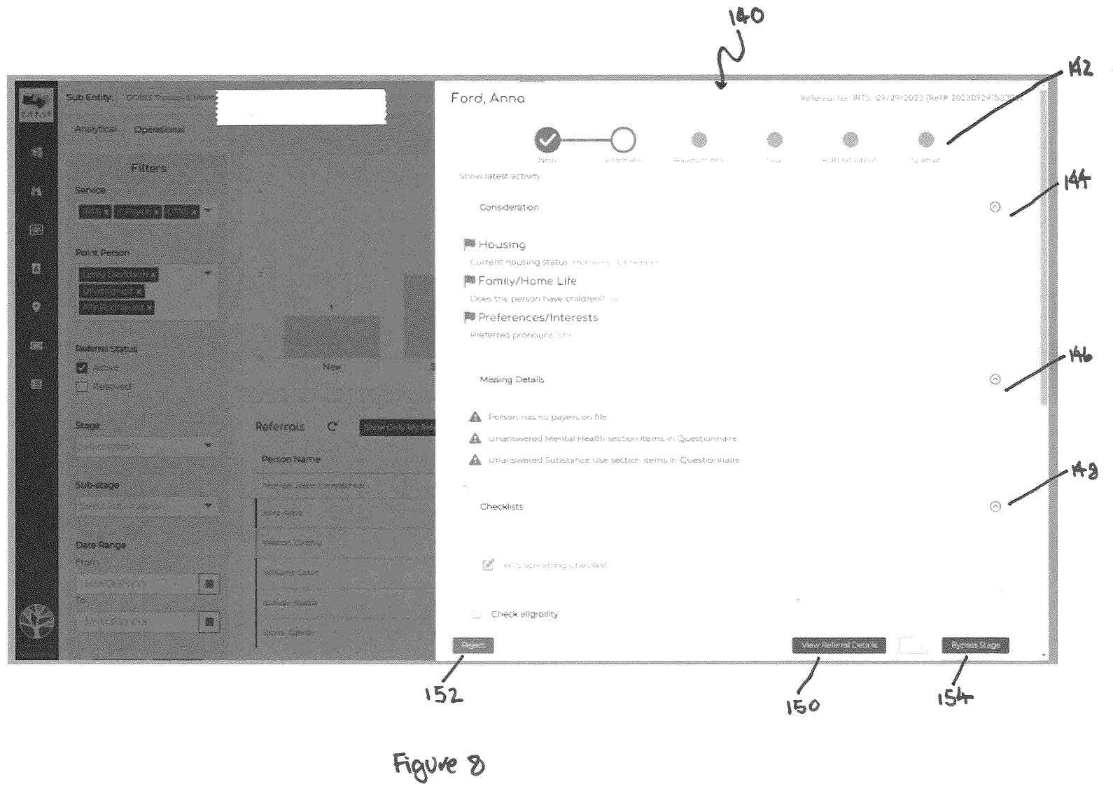

FIG. 8 illustrates a Referral Page 140 that may be displayed for a selected referral that has progressed past the New Stage, in accordance with one embodiment. This page provides a summary of the selected referral and can display various detail about the referral. This may include for example, an Intake Progress bar (progress through the input stages) 142, a Consideration section 144, a Missing Details section 146, and a Checklists section 148. The screen may also give selection options including, for example, View Referral Details 150, Reject 152, or Bypass Stage 154.

The Intake Progress bar 142 may comprise a graphical representation of the status of the referral—showing whether the referral is at the New Stage, the Screening Stage, the Assessment Stage, the Tour Stage, the Authorization Stage, or in the Queue.

The Consideration section 144 may include a summary of entries of interest from a questionnaire (or similar) filled out by or relating to a referral. The system may be configured to compiled this summary based on the values of one or more datapoint selected by an organization or user. During onboarding, a company can configure and tailor the datapoints of interest.

The Reject button 152 allows a user to reject the referral. This can be done at any time after the New Stage. The Bypass Stage button allows a user to bypass the stage. In general, there may be times when a stage is not applicable to a referral based on a real-life exception. If such exception applies, the intake personnel user can bypass the stage in whole or in part.

The Missing Details section 144 may include a list of information that is missing from the referral's record and/or questionnaire. These may include, for example, missing contacts or missing payers, both described more fully below, and missing required details from the questionnaire.

The View Referral Details button allows a user to view and input details of a given referral. FIG. 9 illustrates a Referral Details page or window 160, in accordance with one embodiment. The Referral details page 160 provides a user with the ability to view details about each referral. The Referral Details page 160 may include Further Details sections 162 including, for example, a Person section 164, a Questionnaire section 166, a Payers section 168, a Contacts section 170, an Events section 172, a Logs & Notes section 174, a Referral History section 176, and an Attachments section 178. Each of these sections may be provided directly on the Referral Details page 160 or may be provided on separate windows or pages accessible by clicking on the desired section. The user can select what content to view—for example by selecting a tab associated with the desired section.

FIG. 9 illustrates the Person section 164 opened in the Referral Details page 160 via selection of the Person tab, in accordance with one embodiment. The Person section 164 contains global information categories that apply to all referrals. If the information about a Person is updated in the Person section 164, it will be updated in all referrals linked/associated with that person. The Person section 164 may include referral details such as first name, middle name, last name, preferred name, date of birth, social security number, sex, gender identity, race, and ethnicity. The Person section 164 may further include address (including street, city, state, zip code, county, country, and or other address details), mobile phone, and email address. The Person section 164 may indicate whether the referral has a guardian and/or whether the referral wants a translator. The Person section 164 contains global information about a person that applies to all referrals. If information is changed in the person tab, that information is changed in all referrals linked/associated to the person.

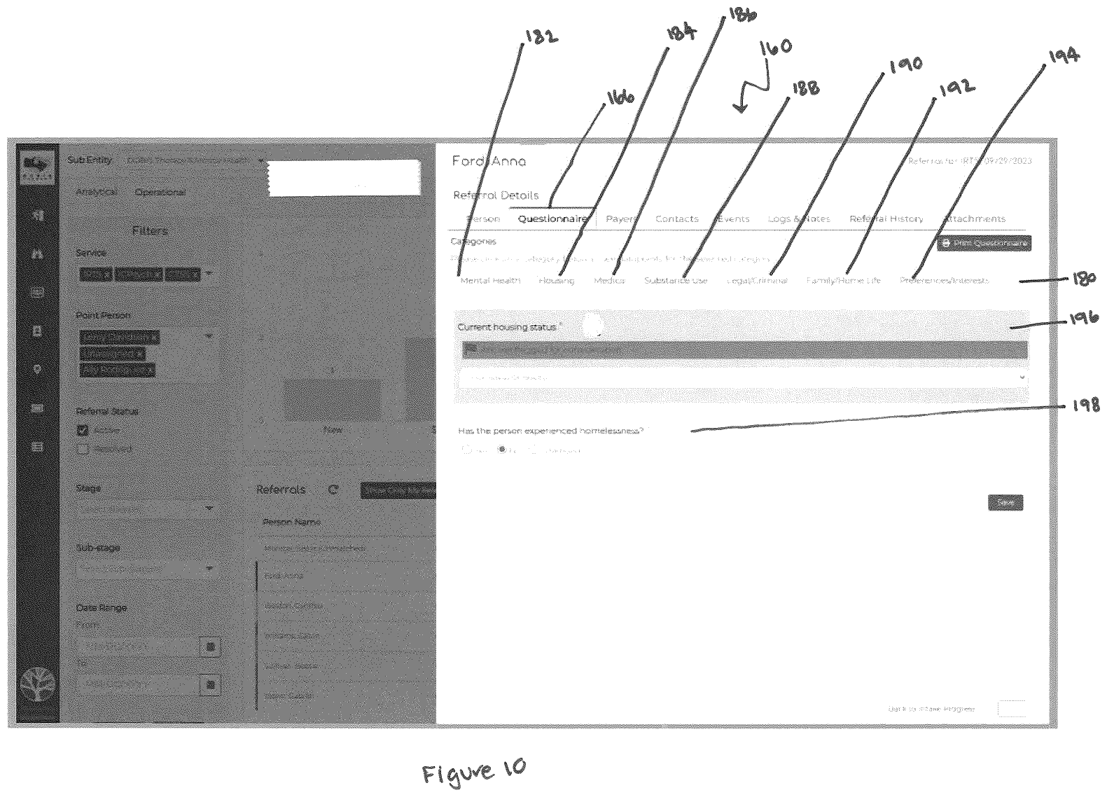

FIG. 10 illustrates the Questionnaire section 166 opened in the Referral Details page 160 via selection of the Questionnaire tab, in accordance with one embodiment. The Questionnaire section may be configured to allow the user to vie and input details about the referral. This may include information relevant to the Consideration section 144 of the Referral Page 140 shown in FIG. 8. The system may be configured to compile this summary based on the values of a data point selected by a user. When an organization company uses the system, the company may customize the data points during onboarding such that the data points are configured and tailored to categories 180 based on the specifications of the company. Organizations may be provided with an expansive and comprehensive menu of datapoints they would like to have displayed. Further, an organization may specify a datapoint not otherwise listed. An organization may designate a datapoint as “required,” “flagged for consideration,” “for information only,” etc. The categories 180 may be associated with tabs, allowing a user easy navigation therethrough.

In the example shown in FIG. 10, the categories 180 in the Questionnaire section 166 include a Mental Health category 182, a Housing category 184, a Medical category 186, a Substance Use category 188 (optionally including a current substance disorder diagnosis and/or current SUD treatment status), a Legal/Criminal category 190, a Family/Home Life category 192, and a Preferences/Interests category 194. Detail from each of these categories 180 may be provided directly on the Questionnaire window 166 of the Referral Details page 160 or may be provided on separate windows or pages accessible by clicking on the desired section. The Questionnaire section 166 may further include information about Current housing status 196 and/or whether the referral has ever experienced homelessness 198.

Datapoints and/or categories in the Questionnaire section are categorized for easy compartmentalization and navigation for a user. When a datapoint meets criteria for consideration, it is flagged in the Questionnaire window 166 and may be added to the Consideration section 144 of the Referral Page 140. Any datapoints defined by an organization as required ma be indicated as such and may be listed on the Missing Details Section 146 of the Referral Page 140.

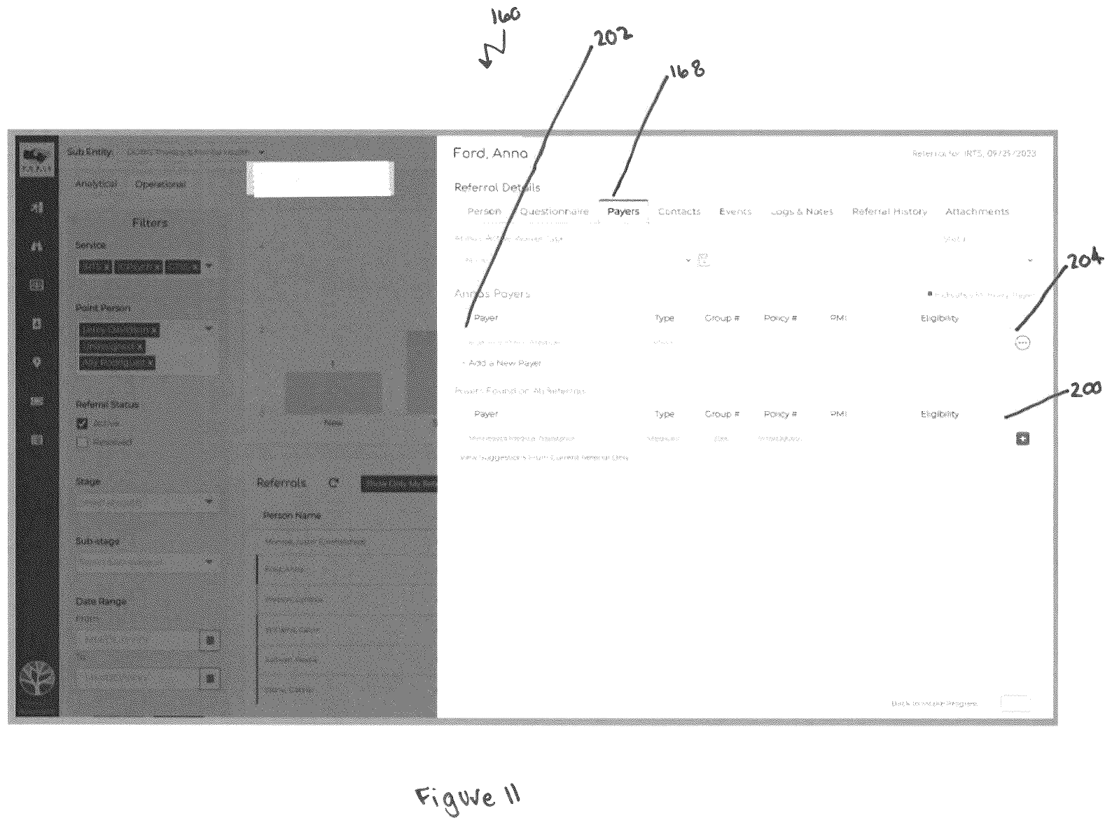

FIG. 11 illustrates the Payers section 168 opened in the Referral Details page 160 via selection of the Questionnaire Payers tab, in accordance with one embodiment. The Payers section organizes the referral's payers in one place. Any payers entered into the referral form appear in the displayed Payers, defaulting to all payers that have ever been entered and associated with the person, shown at 200. This allows the intake personnel user to review all information and process it accordingly into the person's record. Active payers that have been processed into the referral's record may appear at the top of the screen, shown at 202.

A quick-action selection, such as a button or ellipsis 204, may be provided to allow a user to access a menu relating to each payer. This may include the options to view/edit payer information, designate a payer as a primary payer, view/submit eligibility requests, and/or remove/inactivate the payer from the referral's list of payers.

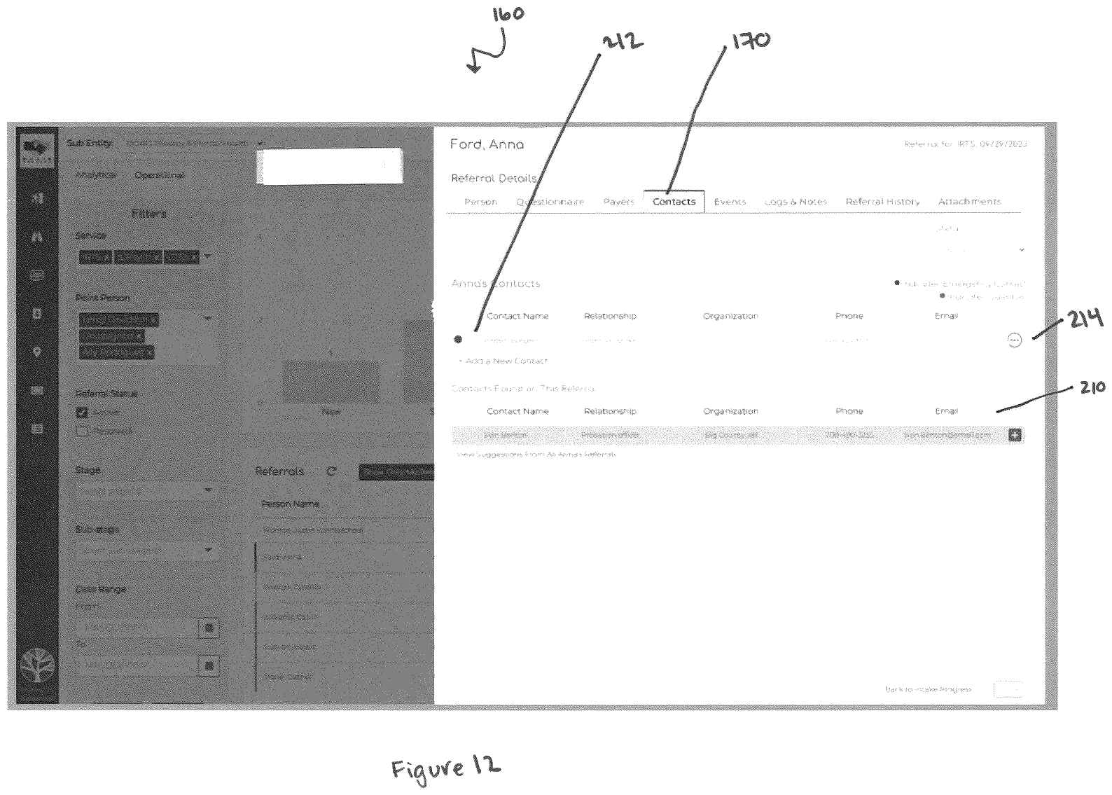

FIG. 12 illustrates the Contacts section 170 open in the Referral Details page 160 via selection of the Contacts tab, in accordance with one embodiment. The Contacts section organizes the referral's contacts in one place. Any contacts entered into the referral form or otherwise submitted for the referral will appear on the Contacts page, defaulting to all contacts that have ever been entered and associated with the referral, shown at 210. This allows the intake personnel user to review all information and process it into the referral's record. The system may be configured to display active contacts that have been processed into the person's record at the top of the screen, shown at 212.

A quick-action selection, such as a button or ellipsis 214, may be provided that allows the user to access a menu relating to each contact. This may include the options to view/edit contact information, designate as emergency contact, and/or remove/inactivate the contact from the referral's list of contacts.

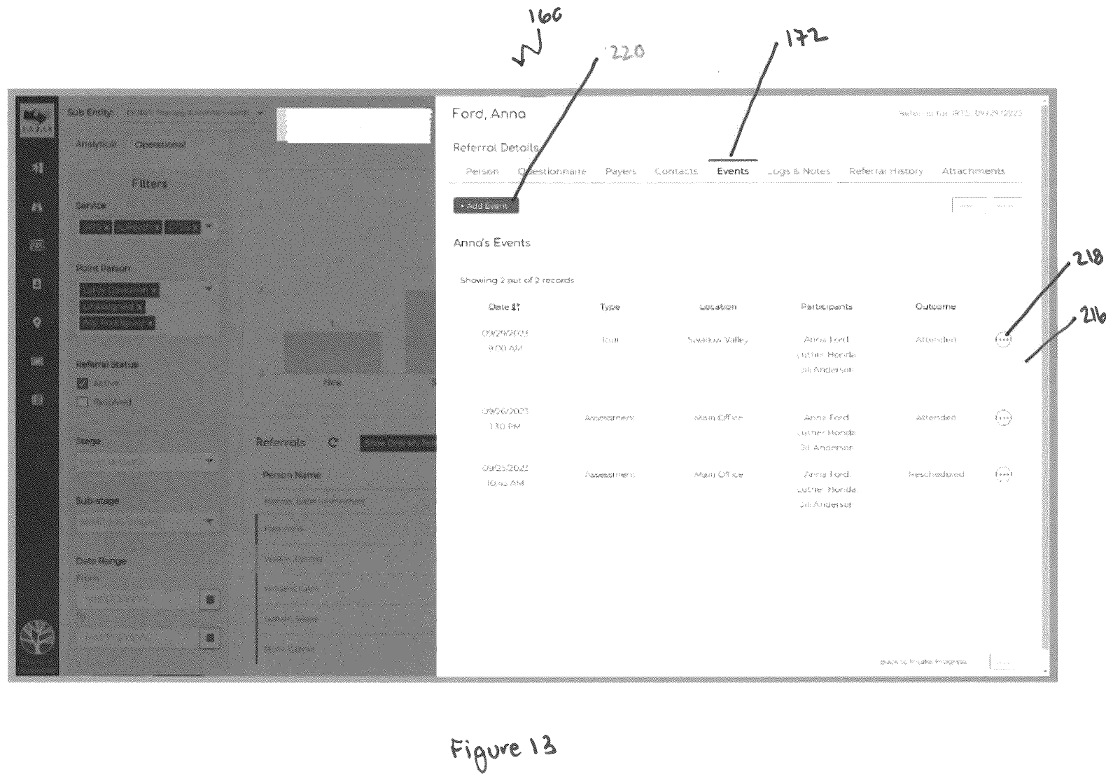

FIG. 13 illustrates the Events section 172 open in the Referral Details page 160 via selection of the Events tab, in accordance with one embodiment. The Events section allows the intake personnel user to view and enter instances of, for example, assessments, tours, placement/admission meetings, and other types of meetings. These events are shown in a list 216. Detail about each event may be provided. Such detail may include the date of the event, the type of event, the location of the invent, the participants of the event, and the outcome of the event. Buttons 218 may be provided to edit the detail about each event. When applicable, an event has the ability to satisfy the requirement as an Assessment, a Tour, or an Admission, each as discussed more fully below.

An intake personnel user can create an event. This may be done on by clicking on an Add Event button 220. Clocking on the Add Event button 220 may open a window that allows the user to enter and record various fields such as those shown in the event details of the list 216, notes, and/or other pertinent information. When an event is created, the system may integrate with other calendar management and meeting platforms such as Microsoft Outlook™, Teams™, Zoom™, etc.

FIG. 14 illustrates the Logs & Notes section 174 open in the Referral Details page 160 via selection of the Logs & Notes tab, in accordance with one embodiment. The Logs & Notes tab allows the intake personnel user to view and enter instances of communication and general notes related to a referral. A list of notes 222 is provided with pertinent details about the notes. This ma include a date, staff name, type, means of communication, and who communication as with. A user can enter a Contact Log 224. This opens a new form that allows the user to choose from the list of contacts and document any communication made with that contact. Alternatively, a user can add a Note 226. This is used for general note taking or internal reference. Any notes or contacts added by the user are added to the list 222.

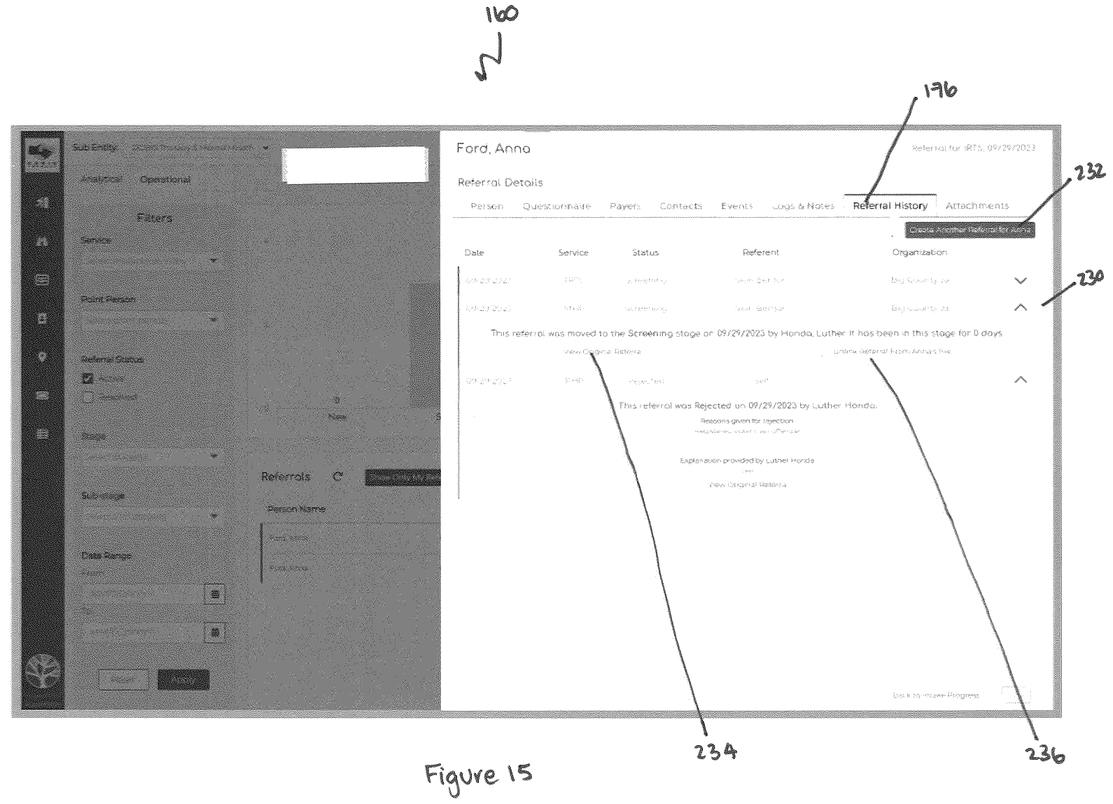

FIG. 15 illustrates the Referral History section 176 open in the Referral Details page 160 via selection of the Referral History tab, in accordance with one embodiment. The Referral History section allows the intake personnel user to see all referrals related to a person as well as details about those referrals such as, date, which service, status, who made the referral, which organization the referent is from, details about the status or outcome of the referral. The Referral History section 176 displays a list of referrals 230 and detail about each referral. Such detail may include, for example, date, service, status, referent, and/or organization.

The intake personnel user may also be given the option to take certain actions. The user may submit a referral directly into the system 222 rather than using a referral form. The user may expedite the internal referral process, with the system auto-generating information for the user to complete the form. The user chooses services from a list and enters a reason for the referral. This also allows for more dynamic analysis and reporting. The user can view the referral in the state it was in when submitted 234, completely unprocessed. The user can unlink the referral from the person's file. 236 The user might do this, for example, if a referral was linked/matched to the wrong person during the New Stage.

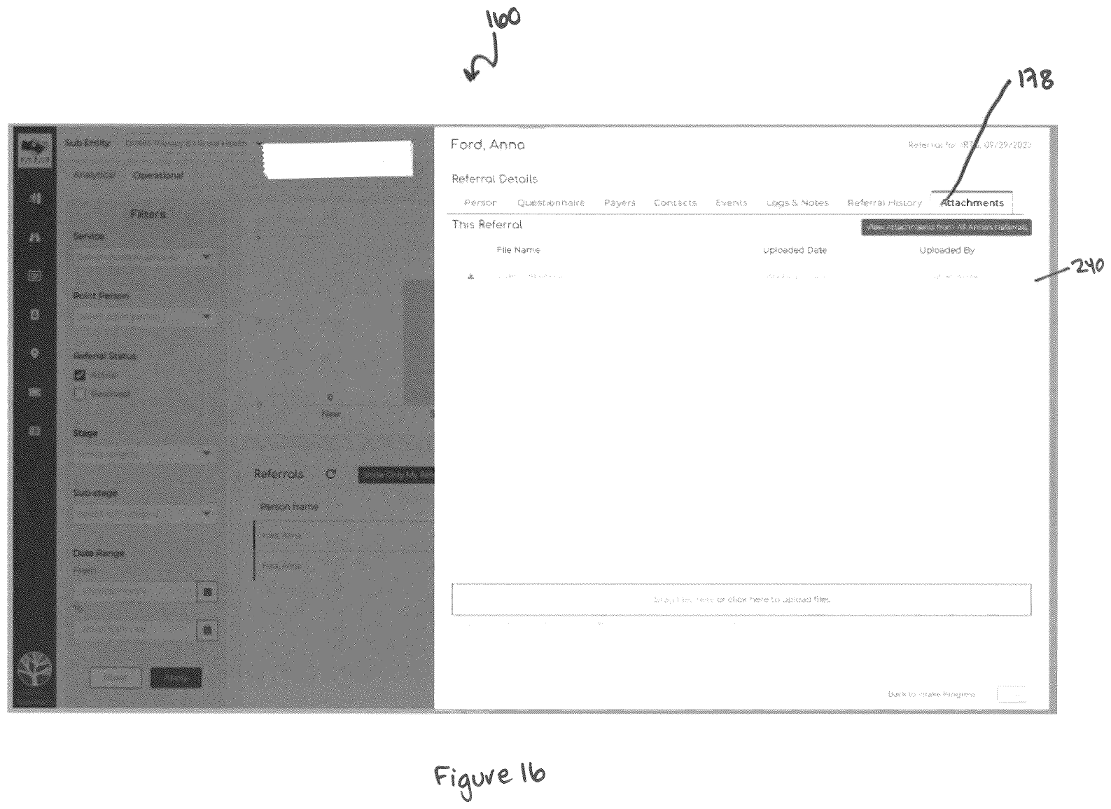

FIG. 16 illustrates the Attachment section 178 open in the Referral Details Attachments section allows the intake personnel user, or other authorized user, to upload files of any external documents they would like to be associated with the referral. These attachments may be specific to the referral, not the person globally, or may be applicable to the person globally. The Attachment section includes a list of attachments 240 and detail about each attachment. Such detail may include file name, upload date, and the name or position of the person who uploaded the file.

For stages subsequent to the Screening stage, including Assessment, Tour, Authorization, and Queue, a second criterion may be introduced. The second criterion may be used to advance the referral through the respective stage.

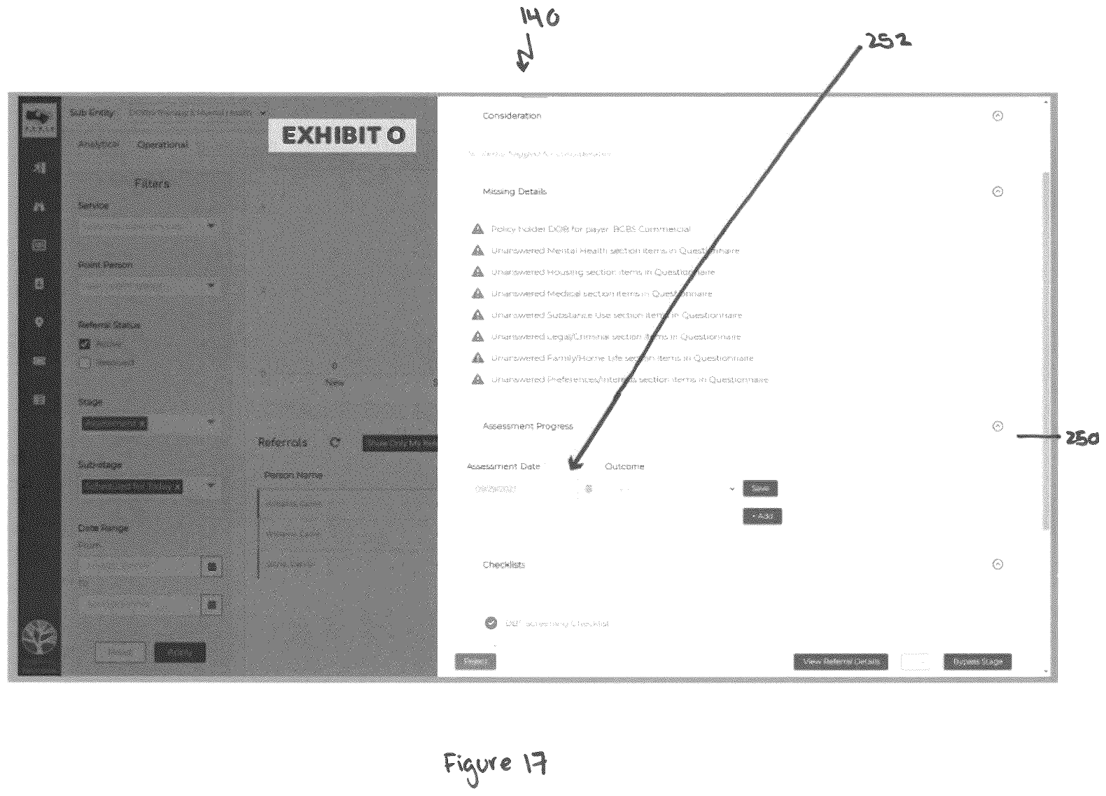

In the Assessment stage the intake personnel user must enter in a date that the person is to be assessed. FIG. 17 illustrates a screen with Assessment Progress. This screen may be a Referral Page 140 such as shown in FIG. 8. The Referral Page 140 may include an Assessment Progress section 250. The user may input information about required assessments 252. This may be via a drop down menu or entered manually. At this point, the user cannot advance the referral until two criteria are met: (1) the checklist is completed and (2) an Assessment date is entered and has an outcome of “Attended.” Should an assessment result in anything other than “Attended,” then the user must enter a subsequent instance of an assessment until it has been attended.

FIG. 18 illustrates a sample page including a Tour section 254, in accordance with one embodiment. The Tour stage is similar to the Assessment stage, except the intake personnel user must also select a location in which the tour is taking place. This allows for deep analysis of frequency of tours by location and success rates of those tours. FIG. 18 further illustrates sample missing details that may be flagged in the assessment stage.

The Authorization stage is similar the Assessment and Tour stages, except additional fields, such as (1) Requested Date, (2) Response Date, and (3) Outcome of “Approved,” may be required.

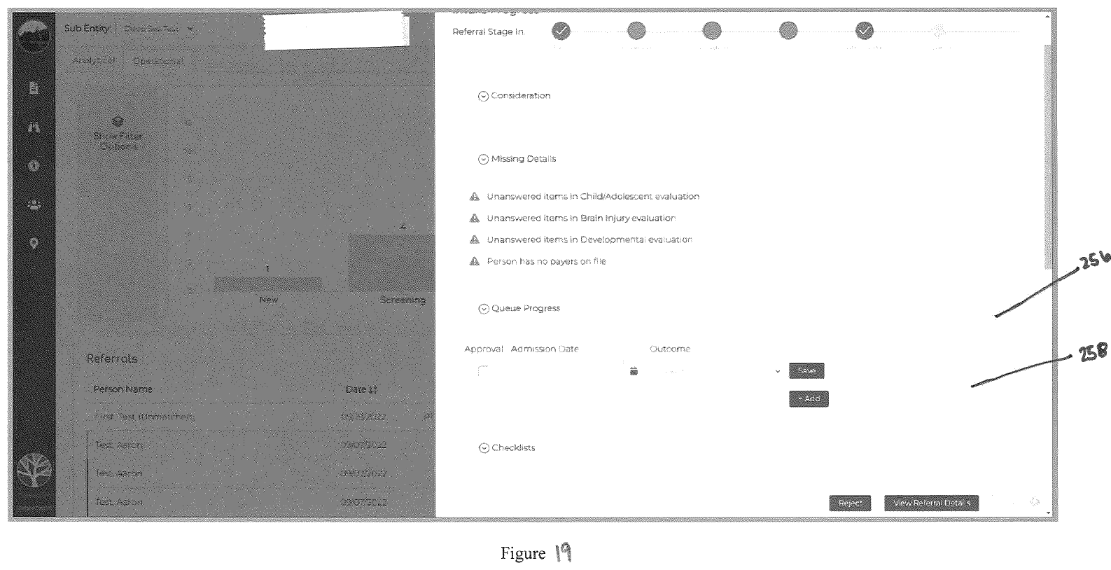

The Queue stage is the final stage. It is similar to previous stages except it may be configured to require approval from a user with assigned permission to do so. This designates a specific task to a person with the authority to ensure all elements of a referral are complete to specification before finally admitting them into a service and thus completing the intake process. After an Admission Date is entered and marked as Attended, a “Complete Intake Process” button appears, which is also controlled by permissions. FIG. 19 illustrates a window including a Queue Progress section 256 and an authorization section 258, in accordance with one embodiment.

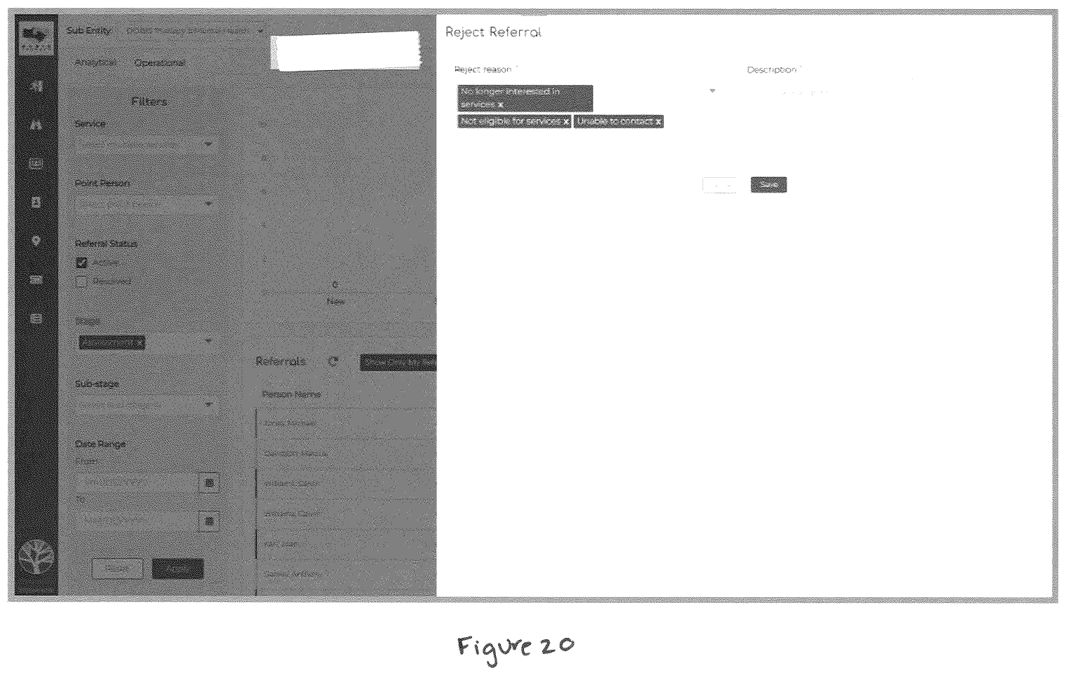

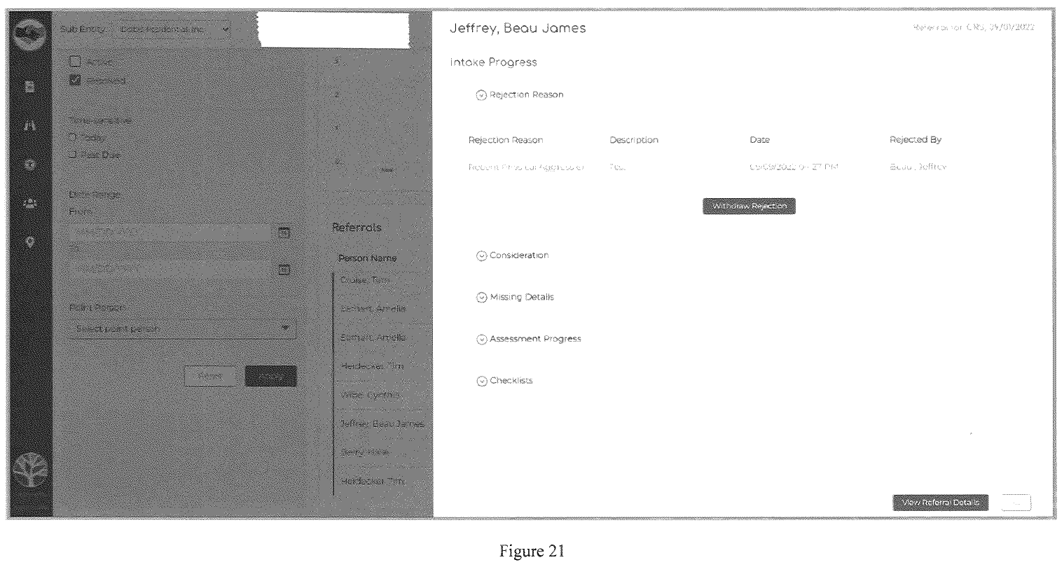

At any time throughout the process, a user can reject a referral. FIG. 20 illustrates a Rejection screen, in accordance with one embodiment. The system will require the user to select from a list of reasons for rejection. They can select more than one reason. These reasons are configured during the onboarding process by service. The user is also required to enter in a description of the rejection reason. In some instances, a rejection may be withdrawn. FIG. 21 illustrates a screen including the option to withdraw a rejection, in accordance with one embodiment.

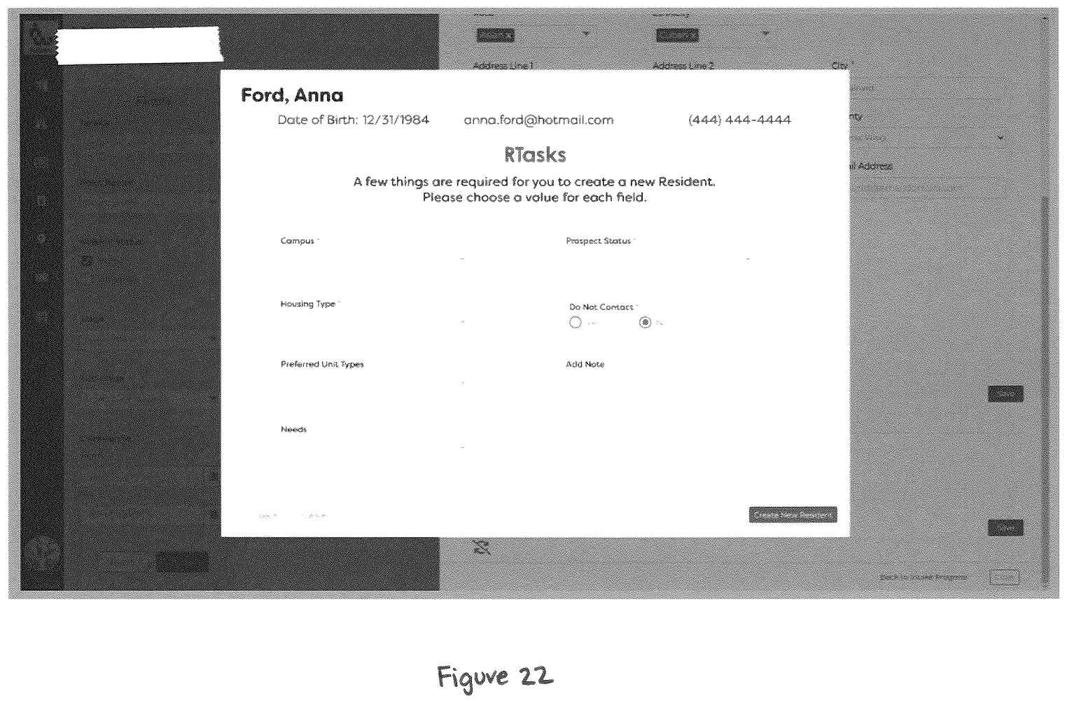

The system may have the ability integrate with a plurality of other platforms, such as Electronic Health/Medical Record (EHR/EMR) systems. When a user is working with a person who has not previously been referred or received service as a client, the user can use the system to push the person's data into the EHR system or any other platforms a customer might use. The user can easily link and synchronize records between the two systems when a client already exists in the integrated platform's database. FIG. 22 illustrates an example screen for integrating a new referral, in accordance with one embodiment.

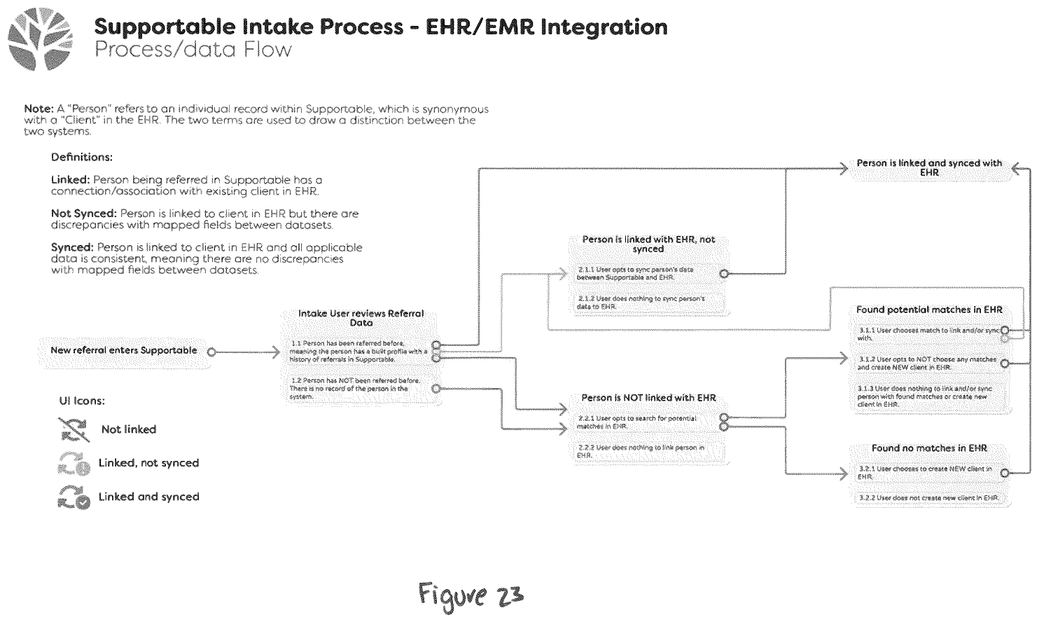

FIG. 23 illustrates general decision tree and data flow between a system for referral management such as disclosed herein and an example EHR system, in accordance with one embodiment.

For purposes of this disclosure, any system described herein may include any instrumentality or aggregate of instrumentalities operable to compute, calculate, determine, classify, process, transmit, receive, retrieve, originate, switch, store, display, communicate, manifest, detect, record, reproduce, handle, or utilize any form of information, intelligence, or data for business, scientific, control, or other purposes. For example, a system or any portion thereof may be a minicomputer, mainframe computer, personal computer (e.g., desktop or laptop), tablet computer, embedded computer, mobile device (e.g., personal digital assistant (PDA) or smart phone) or other hand-held computing device, server (e.g., blade server or rack server), a network storage device, or any other suitable device or combination of devices and may vary in size, shape, performance, functionality, and price. A system may include volatile memory (e.g., random access memory (RAM)), one or more processing resources such as a central processing unit (CPU) or hardware or software control logic, ROM, and/or other types of nonvolatile memory (e.g., EPROM, EEPROM, etc.). A basic input/output system (BIOS) can be stored in the non-volatile memory (e.g., ROM), and may include basic routines facilitating communication of data and signals between components within the system. The volatile memory may additionally include a high-speed RAM, such as static RAM for caching data.

Additional components of a system may include one or more disk drives or one or more mass storage devices, one or more network ports for communicating with external devices as well as various input and output (I/O) devices, such as digital and analog general purpose I/O, a keyboard, a mouse, touchscreen and/or a video display. Mass storage devices may include, but are not limited to, a hard disk drive, floppy disk drive, CD-ROM drive, smart drive, flash drive, or other types of non-volatile data storage, a plurality of storage devices, a storage subsystem, or any combination of storage devices. A storage interface may be provided for interfacing with mass storage devices, for example, a storage subsystem. The storage interface may include any suitable interface technology, such as EIDE, ATA, SATA, and IEEE 1394. A system may include what is referred to as a user interface for interacting with the system, which may generally include a display, mouse or other cursor control device, keyboard, button, touchpad, touch screen, stylus, remote control (such as an infrared remote control), microphone, camera, video recorder, gesture systems (e.g., eye movement, head movement, etc.), speaker, LED, light, joystick, game pad, switch, buzzer, bell, and/or other user input/output device for communicating with one or more users or for entering information into the system. These and other devices for interacting with the system may be connected to the system through I/O device interface(s) via a system bus, but can be connected by other interfaces such as a parallel port, IEEE 1394 serial port, a game port, a USB port, an IR interface, etc. Output devices may include any type of device for presenting information to a user, including but not limited to, a computer monitor, flat-screen display, or other visual display, a printer, and/or speakers or any other device for providing information in audio form, such as a telephone, a plurality of output devices, or any combination of output devices.

A system may also include one or more buses operable to transmit communications between the various hardware components. A system bus may be any of several types of bus structure that can further interconnect, for example, to a memory bus (with or without a memory controller) and/or a peripheral bus (e.g., PCI, PCIe, AGP, LPC, I2C, SPI, USB, etc.) using any of a variety of commercially available bus architectures.

One or more programs or applications, such as a web browser and/or other executable applications, may be stored in one or more of the system data storage devices. Generally, programs may include routines, methods, data structures, other software components, etc., that perform particular tasks or implement particular abstract data types. Programs or applications may be loaded in part or in whole into a main memory or processor during execution by the processor. One or more processors may execute applications or programs to run systems or methods of the present disclosure, or portions thereof, stored as executable programs or program code in the memory, or received from the Internet or other network. Any commercial or freeware web browser or other application capable of retrieving content from a network and displaying pages or screens may be used. In some embodiments, a customized application may be used to access, display, and update information. A user may interact with the system, programs, and data stored thereon or accessible thereto using any one or more of the input and output devices described above.

A system of the present disclosure can operate in a networked environment using logical connections via a wired and/or wireless communications subsystem to one or more networks and/or other computers. Other computers can include, but are not limited to, workstations, servers, routers, personal computers, microprocessor-based entertainment appliances, peer devices, or other common network nodes, and may generally include many or all of the elements described above. Logical connections may include wired and/or wireless connectivity to a local area network (LAN), a wide area network (WAN), hotspot, a global communications network, such as the Internet, and so on. The system may be operable to communicate with wired and/or wireless devices or other processing entities using, for example, radio technologies, such as the IEEE 802.xx family of standards, and includes at least Wi-Fi (wireless fidelity), WiMax, and Bluetooth wireless technologies. Communications can be made via a predefined structure as with a conventional network or via an ad hoc communication between at least two devices.

Hardware and software components of the present disclosure, as discussed herein, may be integral portions of a single computer, server, controller, or message sign, or may be connected parts of a computer network. The hardware and software components may be located within a single location or, in other embodiments, portions of the hardware and software components may be divided among a plurality of locations and connected directly or through a global computer information network, such as the Internet. Accordingly, aspects of the various embodiments of the present disclosure can be practiced in distributed computing environments where certain tasks are performed by remote processing devices that are linked through a communications network. In such a distributed computing environment, program modules may be located in local and/or remote storage and/or memory systems.

As will be appreciated by one of skill in the art, the various embodiments of the present disclosure may be embodied as a method (including, for example, a computer-implemented process, a business process, and/or any other process), apparatus (including, for example, a system, machine, device, computer program product, and/or the like), or a combination of the foregoing. Accordingly, embodiments of the present disclosure may take the form of an entirely hardware embodiment, an entirely software embodiment (including firmware, middleware, microcode, hardware description languages, etc.), or an embodiment combining software and hardware aspects. Furthermore, embodiments of the present disclosure may take the form of a computer program product on a computer-readable medium or computer-readable storage medium, having computer-executable program code embodied in the medium, that define processes or methods described herein. A processor or processors may perform the necessary tasks defined by the computer-executable program code. Computer-executable program code for carrying out operations of embodiments of the present disclosure may be written in an object oriented, scripted or unscripted programming language such as Java, Perl, PHP, Visual Basic, Smalltalk, C++, or the like. However, the computer program code for carrying out operations of embodiments of the present disclosure may also be written in conventional procedural programming languages, such as the C programming language or similar programming languages. A code segment may represent a procedure, a function, a subprogram, a program, a routine, a subroutine, a module, an object, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, etc.

In the context of this document, a computer readable medium may be any medium that can contain, store, communicate, or transport the program for use by or in connection with the systems disclosed herein. The computer-executable program code may be transmitted using any appropriate medium, including but not limited to the Internet, optical fiber cable, radio frequency (RF) signals or other wireless signals, or other mediums. The computer readable medium may be, for example but is not limited to, an electronic, magnetic, optical, electromagnetic, infrared, or semiconductor system, apparatus, or device. More specific examples of suitable computer readable medium include, but are not limited to, an electrical connection having one or more wires or a tangible storage medium such as a portable computer diskette, a hard disk, a random access memory (RAM), a read-only memory (ROM), an erasable programmable read-only memory (EPROM or Flash memory), a compact disc read-only memory (CD-ROM), or other optical or magnetic storage device. Computer-readable media includes, but is not to be confused with, computer-readable storage medium, which is intended to cover all physical, non-transitory, or similar embodiments of computer-readable media.

Various embodiments of the present disclosure may be described herein with reference to flowchart illustrations and/or block diagrams of methods, apparatus (systems), and computer program products. It is understood that each block of the flowchart illustrations and/or block diagrams, and/or combinations of blocks in the flowchart illustrations and/or block diagrams, can be implemented by computer-executable program code portions. These computer-executable program code portions may be provided to a processor of a general purpose computer, special purpose computer, or other programmable data processing apparatus to produce a particular machine, such that the code portions, which execute via the processor of the computer or other programmable data processing apparatus, create mechanisms for implementing the functions/acts specified in the flowchart and/or block diagram block or blocks. Alternatively, computer program implemented steps or acts may be combined with operator or human implemented steps or acts in order to carry out an embodiment of the invention.

Additionally, although a flowchart or block diagram may illustrate a method as comprising sequential steps or a process as having a particular order of operations, many of the steps or operations in the flowchart(s) or block diagram(s) illustrated herein can be performed in parallel or concurrently, and the flowchart(s) or block diagram(s) should be read in the context of the various embodiments of the present disclosure. In addition, the order of the method steps or process operations illustrated in a flowchart or block diagram may be rearranged for some embodiments. Similarly, a method or process illustrated in a flow chart or block diagram could have additional steps or operations not included therein or fewer steps or operations than those shown. Moreover, a method step may correspond to a method, a function, a procedure, a subroutine, a subprogram, etc.

As used herein, the terms “substantially” or “generally” refer to the complete or nearly complete extent or degree of an action, characteristic, property, state, structure, item, or result. For example, an object that is “substantially” or “generally” enclosed would mean that the object is either completely enclosed or nearly completely enclosed. The exact allowable degree of deviation from absolute completeness may in some cases depend on the specific context. However, generally speaking, the nearness of completion will be so as to have generally the same overall result as if absolute and total completion were obtained. The use of “substantially” or “generally” is equally applicable when used in a negative connotation to refer to the complete or near complete lack of an action, characteristic, property, state, structure, item, or result. For example, an element, combination, embodiment, or composition that is “substantially free of” or “generally free of” an element may still actually contain such element as long as there is generally no significant effect thereof.

To aid the Patent Office and any readers of any patent issued on this application in interpreting the claims appended hereto, applicants wish to note that they do not intend any of the appended claims or claim elements to invoke 35 U.S.C. § 112(f) unless the words “means for” or “step for” are explicitly used in the particular claim.

Additionally, as used herein, the phrase “at least one of [X] and [Y],” where X and Y are different components that may be included in an embodiment of the present disclosure, means that the embodiment could include component X without component Y, the embodiment could include the component Y without component X, or the embodiment could include both components X and Y. Similarly, when used with respect to three or more components, such as “at least one of [X], [Y], and [Z],” the phrase means that the embodiment could include any one of the three or more components, any combination or sub-combination of any of the components, or all of the components.

In the foregoing description various embodiments of the present disclosure have been presented for the purpose of illustration and description. They are not intended to be exhaustive or to limit the invention to the precise form disclosed. Obvious modifications or variations are possible in light of the above teachings. The various embodiments were chosen and described to provide the best illustration of the principals of the disclosure and their practical application, and to enable one of ordinary skill in the art to utilize the various embodiments with various modifications as are suited to the particular use contemplated. All such modifications and variations are within the scope of the present disclosure as determined by the appended claims when interpreted in accordance with the breadth they are fairly, legally, and equitably entitled.

What is claimed is:

1. A system for referral management, the system comprising:
a tool configured for one or more users to process a referral through one or more of a plurality of stages wherein the stages include:
a new stage wherein, at the new stage, the referral is associated with a patient record;
a screening stage wherein, at the screening stage, the referral is asked a plurality of screening questions;
an assessment stage wherein, at the assessment stage, a diagnostic assessment of the referral is made;
a tour stage wherein, at the tour stage the referral is given a tour;
an authorization stage wherein, at the authorization stage, the referral is accepted and rejected; and
a queue in which a referral is placed if accepted;
wherein a referral cannot progress from one stage to a next stage unless all required acts in the one stage are performed;
wherein the system includes an active referrals graph and the active referrals graph provides a visual representation of the number of referrals in each stage.

2. The system of claim 1, wherein the active referrals graph further provides a visual representation of the status of each referral in each stage.

3. The system of claim 1, wherein the visual representation is via color coding.

4. The system of claim 1, wherein associating the referral with a patient record comprises associating the referral with an existing client record.

5. The system of claim 1, wherein asking the referral a plurality of screening questions is done via a questionnaire and wherein the questionnaire is customizable by an organization using the system.

6. The system of claim 1, wherein the user inputs identifying credentials when progressing a referral from one stage to a next stage.

7. The system of claim 1, wherein the system tracks an amount of time taken for a referral to progress from one stage to a next stage.

8. The system of claim 1, wherein the screening stage further comprises screening the referral using artificial intelligence.

9. The system of claim 1, further comprising a referrals list associated with the active referrals graph, wherein the referrals list displays detail about each referral.

10. A method for referral management, the method comprising:
receiving patient health information relating to a referral;
inputting the patient health information into a referral management system, the referral management system having a new stage, a screening stage, and an intake stage;
associating the referral with a record during the new stage;
screening the referral during the screening stage;
processing the referral through the intake stage;
accepting or rejecting the referral; and
displaying a visual representation of the stage the referral is in;
wherein the referral cannot advance from one stage to a next stage until all requirements of the one stage are met.

11. The method of claim 10, wherein the intake stage comprises an assessment stage and a tour stage.

12. The method of claim 10, wherein screening is done based by asking a series of questions and wherein the questions are customizable for an organization using the method.

13. The method of claim 10, wherein a user inputs identifying credentials when progressing a referral from one stage to a next stage.

14. The method of claim 10, further comprising tracking an amount of time taken for a referral to progress from one stage to a next stage.

15. The method of claim 10, wherein the visual representation is a bar graph.

16. The method of claim 10, wherein the visual representation is color coded.
